(19) United States
(12) Patent Application Publication
(10) Pub. No.: US 2005/0008639 A1
(43) Pub. Date: Jan. 13, 2005

(54) Methods of modulating homing of T cell by interruption of chemokine/chemokine receptor signaling

(76) Inventors: Alexander V. Chervonsky, Northeast Harbor, ME (US); Alexei Y. Savinov, La Jolla, CA (US)

Correspondence Address: ROPES & GRAY LLP, ONE INTERNATIONAL PLACE, BOSTON, MA, 02110-2624, US

(21) Appl. No.: 10/777,883

(22) Filed: Feb. 12, 2004

Related U.S. Application Data
(60) Provisional application No. 60/447,783, filed on Feb. 14, 2003.

(51) Int. Cl. A61K 39/395

(52) U.S. Cl. 424/145.1; 424/85.1; 514/12

(57) ABSTRACT

In certain embodiments, the invention relates to methods of modulating homing of T cells to the pancreas. Such methods comprise contacting the cells with an agonist or an antagonist of the chemokine CCL21, or with an agonist or an antagonist of a chemokine receptor of the T cells. In other embodiments, the invention relates to methods of treating an individual suffering from insulin-dependent diabetes. Such methods comprise administering to the individual an antagonist of the chemokine CCL21 or a chemokine receptor of the T cells. In yet other embodiments, the invention relates to methods of preventing or reducing the onset of insulin-dependent diabetes in an individual. Such methods comprise administering to the individual an antagonist of the chemokine CCL21 or a chemokine receptor of the T cells.

### RELATED APPLICATIONS

This application claims the benefit of priority of U.S. Provisional Application No. 60/447,783 filed Feb. 14, 2003. The entire teachings of the referenced Provisional Applications are incorporated herein by reference in their entirety.

### FUNDING

Work described herein was supported by National Institutes of Health Grant IDDK53561. The United States Government has certain rights in the invention.

### BACKGROUND

Type I, or insulin-dependent, diabetes mellitus (IDDM) is known to occur spontaneously in humans, rats, and mice. The pathology of type I diabetes consists of the progressive inflammatory infiltration of pancreatic islets (i.e., insulitis) containing immunocytes targeted specifically to insulin-secreting beta-cells. This pathology develops over an indeterminate period of time (months to years). It has become clear that the development of Type I diabetes occurs as a result of a complex relationship involving genetic predisposition, environmental influences, and additional undefined co-factors. However, the immunologic nature of the pathogenic mechanism and the exact antigen(s) inducing the diabetogenic attack have yet to be elucidated.

Over one half million people in the United States suffer from insulin-dependent diabetes. Type I diabetes is a chronic disease that requires life-long treatment to prevent acute illness and to reduce the risk of long-term complications. Restrictive diets and daily insulin injections can be burdensome for patients, thus reducing compliance. Accordingly, more effective treatments for Type I diabetes are needed, in particular, therapies that address the autoimmune basis of the disease, rather than merely treating the symptoms.

### SUMMARY OF THE INVENTION

The present invention relates to methods of modulating homing of T cells to the pancreas as well as methods of treating an individual suffering from insulin-dependent diabetes mellitus (IDDM), through interrupting the signaling through chemokine/chemokine receptor pathway.

In certain embodiments, the invention provides methods of modulating homing of T cells to the pancreas (in particular, the pancreatic islets). In one embodiment, T cells are contacted with an agonist or an antagonist of the chemokine CCL21 in an amount sufficient to modulate homing of T cells to the pancreas. In another embodiment, T cells are contacted with an agonist or an antagonist of a chemokine receptor of the T cells in an amount sufficient to modulate homing of T cells to the pancreas.

In certain embodiments, the invention provides methods of treating an individual suffering from insulin-dependent diabetes. In one embodiment, an individual (patient or subject) suffering from insulin-dependent diabetes is treated by administering to the individual a therapeutically effective amount of an antagonist of the chemokine CCL21. In another embodiment, an individual suffering from insulin-dependent diabetes is treated by administering to the individual a therapeutically effective amount of an antagonist of a chemokine receptor of the T cells. Administration of antagonists of either CCL21 or the chemokine receptor can block homing of T cells to the pancreas and thereby prevent or reduce damage to the insulin-producing β cells. As a result, IDDM is prevented or occurs to a lesser extent (is less severe) than the extent to which it would occur in the absence of such treatment.

As described herein, agonists or antagonists of CCL21 modulate (mimic/enhance or reduce/inhibit, respectively) CCL21 functions. CCL21 functions include, but are not limited to, CCL21 activity (e.g., the ability to interact with a chemokine receptor and to elicit intracellular signaling events) and CCL21 expression level. For example, agonists or antagonists of CCL21 can be an antibody against CCL21, a mutated form or a mimic of CCL21, or small organic molecule or compound such as a peptidomimetic. In one embodiment, agonists or antagonists of CCL21 may modulate CCL21 activity, e.g., modulate the interaction between CCL21 and a chemokine receptor of the T cells. One chemokine receptor for CCL21 is CCR7. Alternatively, the chemokine receptor for CCL21 is CXCR3. In another embodiment, agonists or antagonists of CCL21 may modulate CCL21 expression level, e.g., at the transcriptional level, posttranscriptional level, translational level or posttranslational level.

Similarly, agonists or antagonists of a chemokine receptor modulate (mimic/enhance or reduce/inhibit, respectively) functions of the chemokine receptor. Functions of the chemokine receptor include, but are not limited to, activity of the chemokine receptor (e.g., the ability to interact with CCL21 and to elicit intracellular signaling events) and expression level of the chemokine receptor. For example, agonists or antagonists of the chemokine receptor can be an antibody against the chemokine receptor, a mutated form or a mimic of the chemokine receptor, or small organic molecule or compound such as a peptidomimetic. In one embodiment, agonists or antagonists of the chemokine receptor may modulate activity of the chemokine receptor, e.g., modulate the interaction between CCL21 and the chemokine receptor. One chemokine receptor present on T cells is CCR7. A second chemokine receptor present on T cells is CXCR3. Agonists or antagonists of a chemokine receptor may modulate expression level of the chemokine receptor, e.g., at the transcriptional level, posttranscriptional level, translational level or posttranslational level.

In certain embodiments, the invention provides methods of modulating homing of T cells to the pancreas in an individual. In one embodiment, an individual is administered an agonist or antagonist of the chemokine CCL21 in an amount sufficient to modulate homing of T cells to the pancreas. In another embodiment, an individual is administered an agonist or antagonist of a chemokine receptor (e.g., CCR7 or CXCR3) in an amount sufficient to modulate homing of T cells to the pancreas.

In certain embodiments, the invention provides methods of preventing or reducing the onset of insulin-dependent diabetes in an individual. In one embodiment, an individual is administered an antagonist of CCL21 in an amount effective to prevent or reduce the onset of insulin-dependent diabetes. As a result, IDDM does not occur in the individual or occurs to a lesser extent (is less severe) than would be the case if the treatment were not provided. In another embodiment, an individual is administered an antagonist of a chemokine receptor (e.g., CCR7 or CXCR3) in an amount effective to prevent or reduce the onset of insulin-dependent diabetes.

In all embodiments of methods of treating an individual, one or more antagonists of CCL21 or a chemokine receptor (e.g., CCR7 or CXCR3) can be administered, together (simultaneously) or at different times (sequentially). In addition, antagonists of CCL21 or a chemokine receptor can be administered with another type of compounds for treating insulin-dependent diabetes (e.g., insulin). The two types of compounds may be administered simultaneously or sequentially.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1 shows that pre-existent inflammation is not required for IS-CD8⁺ cells homing into pancreatic islets. (A) IS-CD8⁺ cells cause rapid diabetes in mice that express H2-K^{d}. Recipient mice were irradiated with 725 Rad and injected with 10⁷ IS-CD8⁺ T cells. Diabetes was detected by measuring urine glucose, and confirmed by blood glucose measurement. (B) Cryosections of pancreata from NOD and DBA/2J mice injected with 10⁷ DiI-labeled IS-CD8⁺ cells and sacrificed 24 hrs later. Islet boundaries are marked by a dotted line. For morphometric analysis labeled cells were counted within an area relevant to a given islet (white rectangle). Cells within islet boundaries were considered to be “inside”, cells within rectangle but outside of islet boundaries were considered to be “at the entrance”. (C) IS-CD8⁺ cells can be easily detected in disaggregated pancreatic islets from NOD.Rag1-KO mice 24 hrs after injection. T cells were labeled with CFSE. Numbers indicate percentage of CFSE⁺ CD8⁺ cells in total live cell populations within scatter gates containing (but not limited to) the lymphocyte fraction. (D) Pancreatic islets of NOD.β2m-KO mice contain no CFSE+ IS-CD8⁺ cells 24 hrs after injection. Gates as in (C).

FIG. 2 shows that MHC class 1 expression affects homing of IS-CD8⁺ cells to the pancreatic islets, but not to other tissues. Cryostat sections of the indicated organs isolated 24 hrs after injection of the DiI-labeled IS-CD8⁺ cells. The red color of the tissue is autofluorescence, DiI stained cells are bright yellow. Most typical examples of sections are shown. The red frame highlights the difference in homing of the labeled cells to the pancreata of NOD and NOD.β2m-KO mice.

FIG. 3 shows that lack of specific MHC Class I-peptide complexes affects homing of IS-CD8⁺ cells. (A) In the presence of H2^{k} IS-CD8⁺ cells fail to migrate into islets (a), or kill islet cells (b). Both properties can be explained by the lack of K^{d}-InsB¹⁵⁻²³ complexes. a. Labeled IS-CD8⁺ cells were injected into (C3D2XNOD)F1 mice with either H2^{k/g7} (filled bar) or H2^{d/g7} (clear bar) MHC alleles. Morphometric analysis of the pancreata was performed 24 hrs later. Combined data from two experiments represent mean numbers of IS-CD8⁺ cells per islet (within white rectangle in FIG. 1±SE. n, number of mice per group. *-difference vs. H2^{d/g7} mice is significant by Student's paired t test (p<0.05). b. IS-CD8⁺ cells kill islet cells isolated from H2^{b/d} B6D2 mice, but not the islet cells from H2^{k/d} C3D2 mice. Addition of exogenous InsB¹⁵⁻²³ peptide (done at a 50:1 effector-totarget ratio only) restored killing of H2^{k/d} islet cells to the level of killing of H2^{b/d} islets. Data from a representative experiment. (B) Morphometric analysis of pancreata from β2m-deficient, and -sufficient NOD mice for the presence of labeled IS-CD8⁺ cells at different times after injection. Each cell was ascribed its position as shown in FIG. 1. n, number of mice of each genotype analyzed at each time point. Data show mean numbers of IS-CD8⁺ cells per islet detected at the indicated location ±SE. *-difference between NOD and NOD.β2m-KO is significant by Student's t test (p<0.01).

FIG. 4 shows that endothelial cells from islet organ cultures are killed by IS-CD8⁺ cells in antigen-specific fashion. (A) β cells and endothelial cells can clearly be distinguished both in the native islets (a) and in in vitro culture (b) by staining with anti-Glut-2 (green) and anti-CD105 (red). c, Nomarsky (DIC) image of islet culture (same field as in b). 10× and 20× lenses were used in a and b, c respectively. (B) CD105⁺ cells in the islet cultures are destroyed by IS-CD8⁺ cells in vitro. a. Specific killing of endothelial cells in islet organ cultures from NOD, NOD.β2m-KO, B6.NOD-H2^{g7} and B6 mice by IS-CD8⁺ cells added for 12 hrs on day 7 of islet culture was calculated using the formula: Specific Cytotoxicity (%)=[(mean # of CD105⁺ cells in intact wells-mean # of CD105⁺ cells in the wells exposed to IS-CD8⁺ cells)/mean # of CD105⁺ cells in intact chambers]×100%. CD105⁺ cells (3-4 parallel wells per experiment) were counted using a fluorescent microscope. Combined data from 3 independent experiments. *-difference vs. NOD is significant (p<0.005); **-difference vs. B6.NOD-H2^{g7} is significant (P<0.005) by Student's paired t test. b. Endothelial cells isolated from NOD aorta were killed by IS-CD8⁺ cells in ⁵¹Cr release assay only when exogenous Ins B 5-23 peptide (0.1 μM) was added. Addition of 0.1 μM of control K^{d}-binding LLO⁹¹⁻⁹⁹ peptide had no effect. Data from a representative experiment. (C) IS-CD8⁺ cells do not kill bystander K^{d}-negative CD105⁺ cells in the presence of K^{d} sufficient targets. Islets from NOD and B6-GFP mice were mixed, cultured in vitro for 7 days, then exposed to IS-CD8⁺ cells for 12 hrs or left intact, stained for CD105, fixed and analyzed by fluorescent microscopy. Representative images of NOD and B6-GFP mixed islet cultures before (a), and after exposure to IS-CD8⁺ cells (b). 40× objective was used. c, Formal estimation of the results shown in a and b. Specific loss of GFP-negative, CD105⁺, K^{d}-sufficient NOD cells was determined by the proportion (%) of GFP⁺ CD105⁺ cells among total CD105⁺ cells in mixed NOD and B6-GFP islet cultures left intact or exposed to IS-CD8⁺ cells. Combined data from three independent experiments. *-difference is significant (p<0.05) by Student's t test.

FIG. 5 shows that secretion of insulin is a prerequisite for homing of IS-CD8⁺ cells. Labeled IS-CD8⁺ cells were injected into NOD.B6^{Akita/+} or NOD.B6^{+/+} wild-type littermates. All mice were homozygous for H2^{g}. (A) Morphometric analysis of pancreata was performed at indicated times after injection. Data represent mean numbers of IS-CD8⁺ cells per islet±SE. n, number of mice per group. Difference between groups is significant, as *-p=0.04, **-p=0.03 by Student's t test. (B) Pancreatic islet cells isolated from NOD.B6^{Akita/+} and NOD.B6^{+/+} were sensitive to direct lysis by IS-CD8⁺ effector cells, showing that the cognate MHC-peptide complex was produced by β cells and expressed on their surface. B6^{Akita/+}-K^{d}-negative control. Data from a representative experiment.

FIG. 6 shows that chemokines play an important role in IS-CD8⁺ cells homing to the islets. (A) Treatment of IS-CD8⁺ cells with PTx which blocks signaling through chemokine receptors, abolishes their homing to the islets. IS-CD8⁺ cells treated with PTx in vitro were labeled and injected into NOD mice. In 24 hrs untreated cells penetrated into islets (a), while cells treated with PTx did not (b). c. Morphometric analysis shows quantitative measurement of homing of PTx-treated vs. non-treated IS-CD8⁺ cells. n, number of mice per group. *-p<0.003 by Student's t test. (B) Unlike untreated IS-CD8⁺ cells (a), PTx-treated IS-CD8⁺ cells lost ability to home to T cell zones in the spleen (b), suggesting that SLC may be involved. c. SLC (CCL-21) expression in the splenic T cell zones revealed by staining with specific anti-SLC antibodies (green fluorescence). Serves as positive control to (D). (C) SLC is a primary chemokine responsible for IS-CD8⁺ cells homing to the islets. Pretreatment of recipient mice with anti-SLC serum abolished IS-CD8⁺ cells homing to the islets. Data represent mean number of IS-CD8⁺ cells per islet+SE detected at indicated times after injection. n, number of mice per group. *-p=0.002, **-p=0.001 by Student's t test. (D) SLC expression was found in the isthmus regions of the islets in NOD, DBA/2J and C3D2 mice, indicating that SLC is not the sole factor determining homing of IS-CD8⁺ cells. Negative control included treatment with normal goat serum and secondary labeled antibodies. Islet boundaries are marked by a dotted red line.

FIG. 7 shows that expression of specific MHC-peptide complex induces adhesion of IS-CD8⁺ cells to endothelium and works in concert with SLC. (A) After 300 sec of adhesion to a ortal endothelial cell monolayers pretreated or not with InsB¹⁵⁻²³ peptide, IS-CD8⁺ cells were exposed to shear stress of the flow of tissue culture medium implemented in 4 dyne/cm² increments with 10 sec intervals. *-difference in percentage of adherent IS-CD8⁺ cells is significant for each pair of points of equal shear stress applied (p<0.05 by Student's t test). (B) IS-CD8⁺ cells were pretreated with 100 ng/ml of SLC and allowed to adhere to endothelial monolayers expressing K^{d} complexes with InsB¹⁵⁻²³ or LLO⁹¹⁻⁹⁹ for 90 sec. Inparallel experiments anti-K^{d} monoclonal antibodies were added to monolayers expressing K^{d} complexes with InsB¹⁵⁻²³. *-difference in percentage of adherent IS-CD8⁺ cells is significant for each pair of points of equal shear stress applied (p<0.05 by Student's t test). n, number of measurements.

FIG. 8 provides a model showing how endothelial cells in the pancreas contribute to islet-specific homing of diabetogenic T cells. (A) The vast microcapillary network within the islet of Langerhans revealed by corrosion casting technique suggests an immense surface of interaction between insulin-producing cells and endothelial cells. (B) T cell invasion of the islets requires firm adhesion to endothelial cells. This can be achieved by activation of integrins on the T cell via two independent, but cooperating pathways: via chemokine receptors sensing chemokines deposited on the endothelial cell surface (non-specific component), and via the TCR (specific component). The latter requires presentation of pancreatic antigens such as insulin by endothelial cells. Although it is yet unclear how the cross-presentation occurs, the intact secretion of insulin by β cells is needed.

FIG. 9 shows that irradiation have no effect on pancreatic homing of IS-CD8⁺ cells. NOD.Rag1-KO mice were either irradiated or left intact then injected with DiI-labeled IS-CD8⁺ cells. Morphometric analysis of pancreata was performed 24 hrs after injection. n, number of mice per group.

FIG. 10 shows that homing to the islets requires appropriate specificity of the T cells. (A) A non-islet-specific, CCR7⁺ CD8⁺ T clone Lpa/2R-1 does not migrate to the islets. Dil-labeled IS-CD8⁺ and Lpa/2R-1 cells were injected into NOD recipients. a. Morphometric analysis of pancreata was performed 2 hrs after injection. n, number of mice per group. *-p=0.001, by Student's t test. b. At the same time-point, both IS-CD8⁺ and Lpa/2R-1 cells can be found in lungs in similar numbers. (B) A T cell clone recognizing K^{d} complex with listeria-derived peptide LLO⁹¹⁻⁹⁹ does not home to the islets, but can be found in the NOD spleen 24 hrs after injection of DiI-labeled cells. IS-CD8⁺ cells injected in the same experiment were found both in the islets and in the spleen.

FIG. 11 shows that accumulation of IS-CD8⁺ cells in the islets is not due to local proliferation. FACS profiles of IS-CD8⁺ cells recovered 24 hrs after injection from pancreatic islets (a), and spleens (b) of NOD and NOD.β2m-KO. c. FACS profiles of CFSE-labeled IS-CD8⁺ cells from the same experiment cultured in vitro for 24 and 96 hours.

FIG. 12 shows that triggering of IS-CD8⁺ cells TCR leads to activation of integrins. (A) Anti-CD3 stimulation of IS-CD8⁺ cells leads to an increase in FBN-FITC binding. FACS profiles of FBN-FITC stained CD3-stimulated (3), FBN-FITC stained CD3-unstimulated (2), and BSA-FITC stained (1) IS-CD8⁺ cells. (B) Binding of FBN-FITC by IS-CD8⁺ cells is completely blocked by antibodies to VLA-4 integrin. FACS profiles of IS-CD8⁺ cells stimulated with anti-CD3 and stained with BSA-FITC-negative control (1), and with FBN-FITC in the presence of control anti-VLA-4 antibodies (2), or in the presence of anti-LFA-1 antibodies (3).

### DETAILED DESCRIPTION OF THE INVENTION

During development of insulin-dependent diabetes mellitus (IDDM), autoreactive T cells extravasate from the bloodstream, invade pancreatic islets of Langerhans, and destroy insulin-producing beta cells. CD8⁺ cytotoxic T cells specific to islet antigens, and specifically insulin, are known to play a major role in such destruction. In order to destroy pancreatic islets, CD8⁺ cells must first home into the islets. It is known that T cells home to the secondary lymphoid organs, to specialized compartments such as intestinal epithelium and skin, and also to the sites of inflammation.

The current invention is based, in part, on Applicants' discovery that signaling through chemokine (e.g., CCL21) and a chemokine receptor(s) regulates the islet-specific homing of diabetogenic T cells (e.g., insulin-specific CD8⁺ T cells) and thus contributes to IDDM development. Applicants found that inactivation of G protein-coupled chemokine receptors on diabetogenic insulin-specific CD8⁺ T cells (IS-CD8⁺ cells) with pertussis toxin abolished trafficking of IS-CD8⁺ T cells to the pancreatic islets. Furthermore, signaling caused by a chemokine (e.g., CCL21) was blocked by neutralizing antibodies in vivo, and had a profound effect on the IS-CD8⁺ T cells' ability to adhere to the islet endothelium and to penetrate into the islets. CCL21 expression was detected by immunohistochemical staining at the isthmus of the islets. CCL21 expression was found in the islets of diabetes-prone NOD mice, as well as in the islets of mice with no spontaneous diabetes (DBA/2J or C3D2 mice).

Chemokines (chemoattractant cytokines) comprise a family of structurally related secreted proteins that share the ability to induce migration and activation of specific types of blood cells (reviewed in Baggiolini M., et al. (1997) Annu. Rev. inmmunol. 15: 675-705; Yoshie, et al. (1997) J. Leukocyte Biol. 62: 634-644). Chemokines vary in their specificities for different leukocyte types, and in the types of cells and tissues where the chemokines are synthesized. For example, some chemokines act selectively on immune system cells such as subsets of T-cells or B lymphocytes or antigen presenting cells, and may thereby promote immune responses to antigens. The activities of chemokines are mediated by cell surface receptors which are members of the family of seven transmembrane, G-protein coupled receptors. These chemokine receptors vary in their specificites for specific chemokines. Binding of a chemokine to its receptor typically induces intracellular signaling responses such as a transient rise in cytosolic calcium concentration, followed by cellular biological responses such as chemotaxis.

The chemokine CCL21 is also referred to in the literature as exodus-2, beta chemokine exodus-2, 6Ckine, secondary lymphoid tissue chemokine (SLC), and small inducible cytokine subfamily A, member 21. Previously, CCL21 has been known for its ability to bind to CCR7 and CXCR3 chemokine receptors on the surface of T cells. It has been shown to promote homing of T cells to T cell zones of spleen and to lymph nodes. It has also been shown to trigger β2 integrin affinity and mobility changes promoting lymphocyte adhesion to the endothelial wall during extravasation process.

In certain embodiments, the present invention provides methods of modulating homing of T cells to the pancreas, in particular, to the pancreatic islets. In one embodiment, T cells are contacted with an agonist or an antagonist of the chemokine CCL21 in an amount sufficient to modulate homing of T cells to the pancreas. In another embodiment, T cells are contacted with an agonist or an antagonist of a chemokine receptor of the T cells in an amount sufficient to modulate homing of T cells to the pancreas.

In these embodiments, agonists of CCL21 include compounds (agents) which mimic or enhance functions of CCL21, while antagonists of CCL21 include compounds (agents) which reduce or inhibit functions of CCL21. Functions of CCL21 include, but are not limited to, CCL21 activity (e.g., the ability to interact with a chemokine receptor and to elicit intracellular signaling events) and CCL21 expression level. For example, agonists or antagonists of CCL21 can be an antibody against CCL21, a mutated form or a mimic of CCL21, or a peptidomimetic. In one embodiment, agonists or antagonists of CCL21 may modulate CCL21 activity, e.g., modulate the interaction between CCL21 and a chemokine receptor of the T cells. One chemokine receptor (present on T cells) for CCL21 is CCR7. A second chemokine receptor (present on T cells) for CCL21 is CXCR3. In another embodiment, agonists or antagonists of CCL21 may modulate CCL21 expression level, e.g., at the transcriptional level, posttranscriptional level, translational level or posttranslational level.

Similarly, agonists of a chemokine receptor include compounds (agents) which mimic or enhance the function of the chemokine receptor, while antagonists of a chemokine receptor include compounds (agents) that reduce or inhibit functions of the chemokine receptor. Functions of the chemokine receptor include, but are not limited to, activity of the chemokine receptor (e.g., the ability to interact with CCL21 and to elicit intracellular signaling events) and expression level of the chemokine receptor. For example, agonists or antagonists of the chemokine receptor can be an antibody against the chemokine receptor, a mutated form or a mimic of the chemokine receptor, or a peptidomimetic. In one embodiment, agonists or antagonists of the chemokine receptor may modulate activity of the chemokine receptor, e.g., modulate the interaction between CCL21 and the chemokine receptor. One chemokine receptor present on T cells is CCR7. A second chemokine receptor present on T cells is CXCR3. In another embodiment, agonists or antagonists of the chemokine receptor may modulate expression level of the chemokine receptor, e.g., at the transcriptional level, posttranscriptional level, translational level or posttranslational level.

In certain embodiments, the invention provides methods of modulating homing of T cells to the pancreas in an individual. In one embodiment, an individual is administered an agonist or antagonist of the chemokine CCL21 in an amount sufficient to modulate homing of T cells to the pancreas. In another embodiment, an individual is administered an agonist or antagonist of a chemokine receptor (e.g., CCR7 or CXCR3) in an amount sufficient to modulate homing of T cells to the pancreas.

In certain embodiments, the invention provides methods of treating an individual suffering from insulin-dependent diabetes. In one embodiment, an individual (patient or subject) suffering from insulin-dependent diabetes is treated by administering to the individual a therapeutically effective amount of an antagonist of CCL21. In another embodiment, an individual suffering from insulin-dependent diabetes is treated by administering to the individual a therapeutically effective amount of an antagonist of a chemokine receptor of the T cells (e.g., CCR7 or CXCR3). Administration of antagonists of either CCL21 or the chemokine receptor can block homing of T cells to the pancreas (in particular, the pancreatic islets) and thereby prevent damage to the insulin-producing β cells.

In these embodiments, antagonists of CCL21 reduce or inhibit functions of CCL21. Functions of CCL21 include, but are not limited to, CCL21 activity (e.g., the ability to interact with a chemokine receptor and to elicit intracellular signaling events) and CCL21 expression level. For example, antagonists of CCL21 can be an antibody against CCL21, a mutated form or a mimic of CCL21, or a peptidomimetic. In one embodiment, the CCL21 antagonists may reduce or inhibit CCL21 activity, e.g., the interaction between CCL21 and a chemokine receptor of the T cells. One chemokine receptor (present on T cells) for CCL21 is CCR7. A second chemokine receptor (present on T cells) is CXCR3. In another embodiment, the CCL21 antagonists may reduce or inhibit CCL21 expression level, e.g., at the transcriptional level, posttranscriptional level, translational level or posttranslational level.

Similarly, antagonists of a chemokine receptor of T cells reduce or inhibit functions of the chemokine receptor. Functions of the chemokine receptor include, but are not limited to, activity of the chemokine receptor (e.g., the ability to interact with CCL21 and to elicit intracellular signaling events) and expression level of the chemokine receptor. For example, antagonists of the chemokine receptor can be an antibody against the chemokine receptor, a mutated form or a mimic of the chemokine receptor, or a peptidomimetic. In one embodiment, antagonists of the chemokine receptor may reduce or inhibit activity of the chemokine receptor, e.g., the interaction between CCL21 and the chemokine receptor. One chemokine receptor present on T cells is CCR7. A second chemokine receptor (present on T cells) is CXCR3. In another embodiment, antagonists of the chemokine receptor may reduce or inhibit expression level of the chemokine receptor, e.g., at the transcriptional level, posttranscriptional level, translational level or posttranslational level.

In yet certain embodiments, the invention provides methods of preventing or reducing the onset of insulin-dependent diabetes in an individual. For example, an individual who is at risk of developing IDDM (e.g. an individual whose family history includes IDDM) and/or has signs he/she will develop IDDM (e.g., elevated blood glucose levels) can be treated by the present methods. The methods of the present can be used prophylactically to prevent the onset of IDDM or reduce the extent to which it occurs. In one embodiment, an individual is administered an antagonist of CCL21 in an amount effective to prevent or reduce the onset of insulin-dependent diabetes. In another embodiment, an individual is administered an antagonist of a chemokine receptor (e.g., CCR7 or CXCR3) in an amount effective to prevent or reduce the onset of insulin-dependent diabetes. In these embodiments, the antagonist may be administered as a prophylactic agent. Homing of T cells to the pancreas can be slowed early enough to prevent any permanent damage to the insulin-producing β cells.

### Agonists and Antagonists

As used herein, agonists and antagonists of either CCL21 or a chemokine receptor (e.g., CCR7 or CXCR3) include any compound (agent) which modulates functions of CCL21 or the chemokine receptor, such as a protein, peptide, small organic molecule, nucleic acid, peptidomimetic, soluble chemokine receptor, and antibody.

For example, antagonists of CCL21 include an antibody which binds to CCL21 and inhibits the interaction between CCL21 and a chemokine receptor, an agent (e.g., a fragment of CCL21) which binds to the chemokine receptor but does not elicit intracellular signaling events, and a compound which reduces or inhibits the CCL21 expression. Similarly, exemplary antagonists of a chemokine receptor includes an antibody which binds to the chemokine receptor and inhibits the interaction between the chemokine receptor and CCL21, an agent (e.g., a fragment of the chemokine receptor) which binds to CCL21 and prevents the interaction between CCL21 and the wild-type chemokine receptor, and a compound which reduces or inhibits the chemokine receptor expression.

In certain embodiments, antibodies are exemplary agonists or antagonists. Antibodies may be polyclonal or monoclonal; intact or truncated, e.g., F(ab′)₂, Fab, Fv; xenogeneic, allogeneic, syngeneic, or modified forms thereof, e.g., humanized, chimeric, etc. Antibodies generation against CCL21 or a chemokine receptor (e.g., CCR7 or CXCR3) polypeptide can be obtained by administering the polypeptide or epitope-bearing fragments, analogs or cells to an animal, preferably a nonhuman, using routine protocols. For preparation of monoclonal antibodies, any technique which provides antibodies produced by continuous cell line cultures can be used. Examples include the hybridoma technique (Kohler, et al., Nature (1975) 256:495-497), the trioma technique, the human B-cell hybridoma technique (Kozbor, et al., Immunology Today (1983) 4:72), and the EBV-hybridoma technique (Cole, et al., Monoclonal Antibodies And Cancer Therapy, pp. 77-96, Alan R. Liss, Inc., 1985). Techniques for the production of single chain antibodies (US Patent No. 4,946,778) can also be adapted to produce single chain antibodies (e.g., against CCL21 or a chemokine receptor). Also, transgenic mice or other organisms including other mammals, may be used to express humanized antibodies.

Potential agonists or antagonists may include a small molecule (such as a peptidomimetic) that binds to CCL21 or a chemokine receptor, making it either more readily accessible or inaccessible to the other binding partner such that normal biological activity is enhanced or prevented. Examples of small molecules include, but are not limited to, small peptides or peptide-like molecules (e.g., a peptidomimetic). As used herein, the term “peptidomimetic” includes chemically modified peptides and peptide-like molecules that contain non-naturally occurring amino acids, peptoids, and the like. Peptidomimetics provide various advantages over a peptide, including enhanced stability when administered to a subject. Methods for identifying a peptidomimetic are well known in the art and include the screening of databases that contain libraries of potential peptidomimetics. For example, the Cambridge Structural Database contains a collection of greater than 300,000 compounds that have known crystal structures (Allen et al., Acta Crystallogr. Section B, 35:2331 (1979)). Where no crystal structure of a target molecule is available, a structure can be generated using, for example, the program CONCORD (Rusinko et al., J. Chem. Inf. Comput. Sci. 29:251 (1989)). Another database, the Available Chemicals Directory (Molecular Design Limited, Informations Systems; San Leandro Calif.), contains about 100,000 compounds that are commercially available and also can be searched to identify potential peptidomimetics of CCL21 or a chemokine receptor.

In particular, potential antagonists also include soluble forms of a chemokine receptor (e.g., CCR7 or CXCR3), such as fragments of the receptor which bind to CCL21 and prevent CCL21 from interacting with membrane bound (wild-type) chemokine receptor.

In certain embodiments, agonists and antagonists also encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl, sulfhydryl or carboxyl group.

Candidate agonists and antagonists can be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant, and animal extracts are available or readily produced. Additionally, natural or synthetically produced libraries and compounds can be modified through conventional chemical, physical, and biochemical means. Known pharmacological agents may be subjected to directed or random chemical modifications, such as acylation, alkylation, esterification, and amidification, to produce structural analogs.

The present invention also contemplates agonists and antagonists obtainable from the screening methods described as below.

### Screening Assays

Methods of the present invention also employ agonists or antagonists which can be identified by a variety of screening methods. Such agonists or antagonists either stimulate or inhibit functions (e.g., activity or expression level) of CCL21 or a chemokine receptor. Preferably, antagonists are employed for therapeutic and prophylactic purposes for insulin-dependent diabetes, while both agonists and antagonists are employed for modulating homing of T cells to the pancreatic islets.

In general, such screening procedures involve providing appropriate cells that express a chemokine receptor (e.g., CCR7 or CXCR3) on the surface thereof. Such cells include cells from mammals, yeast, Drosophila, and E. coli. In particular, a polynucleotide encoding the chemokine receptor is employed to transfect cells to thereby express the chemokine receptor. The cell expressing the chemokine receptor or the expressed chemokine receptor is then contacted with a test compound (agent) to observe binding, stimulation or inhibition of a functional response, or expression (protein or nucleic acid).

An exemplary screening procedure involves the use of melanophores that are transfected to express a chemokine receptor. Such a screening technique is described in PCT WO 92/01810. This assay may be employed to screen for compounds which inhibit signaling of the chemokine receptor by contacting the melanophore cells expressing the receptor with both the receptor ligand (e.g., CCL21), and a compound to be screened. Inhibition of the signal generated by CCL21 indicates that a compound is a potential antagonist of the chemokine receptor or CCL21.

In other cases, the technique may also be employed for screening of compounds that activate signaling of the chemokine receptor by contacting the melanophore cells with compounds to be screened and determining whether such a compound generates a signal. Activation of the signal generated by the compound indicates that the compound is a potential agonist of CCL21.

Other screening techniques include the use of cells that express a chemokine receptor (e.g., transfected CHO cells) in a system which measures a second messenger response, for example, changes of extracellular pH, cAMP, calcium, proton or other ions, caused by the chemokine receptor activation. In this technique, compounds may be contacted with cells expressing the chemokine receptor. A second messenger response is then measured to determine whether the potential compound activates or inhibits signaling through the chemokine receptor.

Another method involves screening for antagonist compounds by determining inhibition of binding of labeled CCL21 to cells which have a chemokine receptor (e.g., CCR7 or CXCR3) on the surface thereof, or cell membranes containing the receptor. This method involves transfecting a eukaryotic cell with DNA encoding a chemokine receptor. The cell is then contacted with a potential antagonist compound in the presence of a labeled CCL21 (e.g., radiolabeled). The amount of labeled CCL21 bound to the receptors is measured, e.g., by measuring radioactivity associated with transfected cells or membrane from these cells. If the compound binds to the receptor, the binding of labeled CCL21 to the receptor is inhibited as determined by a reduction of labeled CCL21 which binds to the receptor. This method is called binding assay. Naturally, this same technique can be used to screen for an agonist compound which enhances binding between CCL21 and the chemokine receptor.

A functional assay that detects T cell homing to pancreatic islets (e.g., adhesion to the islet endothelium) may be used for screening for agonists or antagonists. For example, T cells (e.g., CD8⁺ T cells) may be stimulated with CCL21, in the presence or absence of the candidate compound (agent). An antagonist that blocks signaling through CCL21/chemokine receptor will cause a decrease in the T cell adhesion to the islet endothelial cell. On the other hand, an agent that is an agonist will increase adhesion of the T cells to the islet endothelial cell, either in the absence or in the presence of CCL21.

Other screening assays that detect the expression level (protein or nucleic acid) of CCL21 or a chemokine receptor (CCR7 or CXCR3) may be used for screening for agonists or antagonists. Methods of detecting and optionally quantitating proteins can be achieved by techniques such as antibody-based detection assays. In these cases, antibodies may be used in a variety of detection techniques, including enzyme-linked immunosorbent assays (ELISAs), immunoprecipitations, and Western blots. On the other hand, methods of detecting and optionally quantitating nucleic acids generally involve preparing purified nucleic acids and subjecting the nucleic acids to a direct detection assay or an amplification process followed by a detection assay. Amplification may be achieved, for example, by polymerase chain reaction (PCR), reverse transcriptase (RT), and coupled RT-PCR. Detection of nucleic acids is generally accomplished by probing the purified nucleic acids with a probe that hybridizes to the nucleic acids of interest, and in many instances, detection involves an amplification as well. Northern blots, dot blots, microarrays, quantitative PCR, and quantitative RT-PCR are all well known methods for detecting nucleic acids.

### Methods of Treatment

In certain embodiments, the present invention provides methods of treating an individual suffering from insulin-dependent diabetes through administering to the individual a therapeutically effective amount of an antagonist of CCL21 or an antagonist of a chemokine receptor as described above. In other embodiments, the invention provides methods of preventing or reducing the onset of insulin-dependent diabetes in an individual through administering to the individual an effective amount of an antagonist of CCL21 or an antagonist of a chemokine receptor. These methods are particularly aimed at therapeutic and prophylactic treatments of animals, and more particularly, humans.

In certain embodiments, methods of the invention are directed to reducing both activities and amounts of CCL21 or a chemokine receptor, in an individual suffering from insulin-dependent diabetes. If the activity of CCL21 or a chemokine receptor is in excess, several approaches are available. For example, one approach comprises administering to a subject an antagonist as hereinabove described along with a pharmaceutically acceptable carrier in an amount effective to inhibit activation by blocking binding of CCL21 to a chemokine receptor, or by inhibiting a second signal, and thereby blocking homing of T cells to the pancreas and preventing damage to the insulin-producing β cells.

In certain embodiments of such methods, one or more antagonists of CCL21 or a chemokine receptor can be administered, together (simultaneously) or at different times (sequentially). In addition, antagonists of CCL21 or a chemokine receptor can be administered with another type of compounds for treating insulin-dependent diabetes (e.g., insulin). The two types of compounds may be administered simultaneously or sequentially.

In certain embodiments, gene therapy may be applicable with the use of nucleic acids encoding antagonist polypeptides (for example, fragments of CCL21 or a chemokine receptor) or an antisense nucleic acid which can reduce or inhibit expression of CCL21 or the chemokine receptor. Preferably, such gene therapy is tissue-specific for T cells or the pancreatic islets.

### Formulation and Administration

In certain embodiments of the present invention, agonists and antagonists may be formulated in combination with a suitable pharmaceutical carrier. Such formulations comprise a therapeutically effective amount of the agonists or antagonist, and a pharmaceutically acceptable carrier (excipient). Such carriers include, but are not limited to, saline, buffered saline, dextrose, water, glycerol, ethanol, and combinations thereof. Formulation should suit the mode of administration, and is well within the skill of the art.

Agonists or antagonists may be employed alone or in conjunction with other compounds, such as a therapeutic compound (e.g., insulin) for treating insulin-dependent diabetes. These different types of compounds may be administered in the same formulation or in a separate formulation.

Preferred forms of systemic administration of the pharmaceutical compositions include injection, typically by intravenous injection. Other injection routes, such as subcutaneous, intramuscular or intraperitoneal, can be used. Alternative means for systemic administration include transmucosal and transdermal administration using penetrants such as bile salts or fusidic acids or other detergents. In addition, if properly formulated in enteric or encapsulated formulations, oral administration may also be possible. Administration of these compounds may also be topical and/or localized, in the form of salves, pastes, gels, and the like.

The dosage range depends on the choice of peptide, the route of administration, the nature of the formulation, the nature of the subject's condition, and the judgment of the attending practitioner. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

### Exemplification

The invention now being generally described, it will be more readily understood by reference to the following examples, which are included merely for purposes of illustration of certain embodiments and embodiments of the present invention, and are not intended to limit the invention. Homing of the IS-CD8⁺ cells to the pancreatic islets does not require pre-existent inflammation.

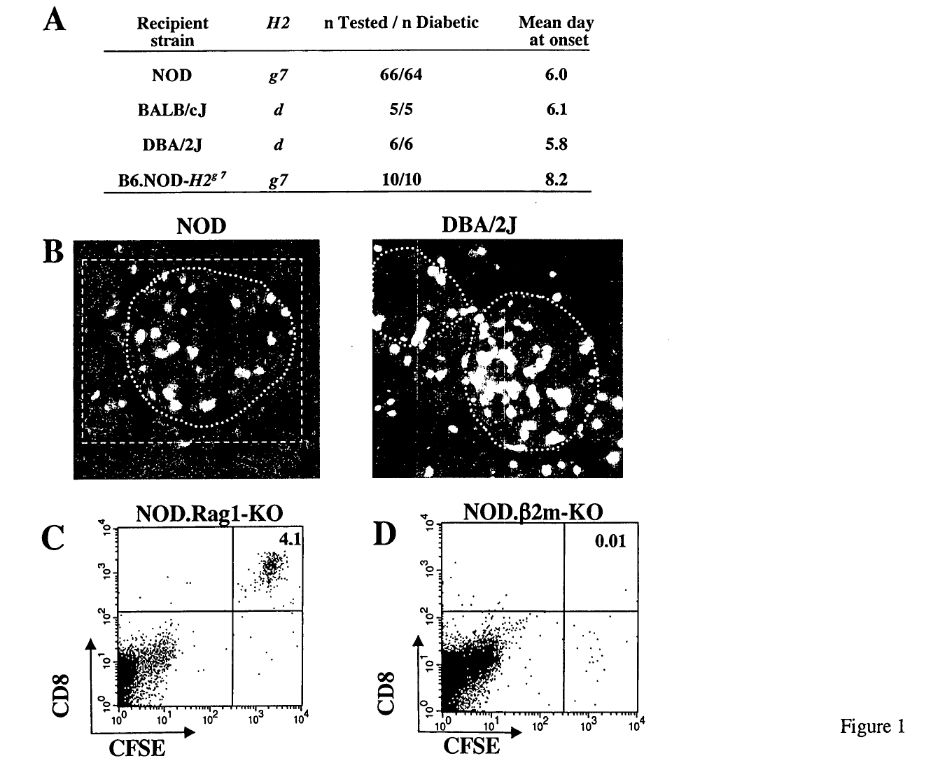

To address the question of whether the homing of IS-CD8⁺ cells into the pancreas requires an ongoing inflammation that induces expression of adhesion molecules and chemokines, we performed adoptive transfer experiments. IS-CD8⁺ cells were injected i.v. into NOD/LtJ, BALB/cJ, and DBA/2J mice and caused rapid diabetes in all strains tested (FIG. 1A). All of these mice express MHC class I K^{d} molecules. Strains that do not express K^{d} were previously shown to be resistant (6). We also performed a direct in vivo tracing experiment. For that, IS-CD8⁺ cells were labeled with a fluorescent dye, DiI, and injected i.v. into NOD and DBA/2J recipients. 24 hours later the pancreata were fixed and cryostat sections analyzed. Labeled IS-CD8⁺ cells were readily detected in the islets in both NOD and DBA/2J mice (FIG. 1B). In addition we injected labeled IS-CD8⁺ cells into NOD.Rag1-KO mice, which lack T and B lymphocytes, and are inflammation-free. FIG. 1C shows that IS-CD8⁺ cells could be easily found in the islets of NOD.Rag1-KO mice.

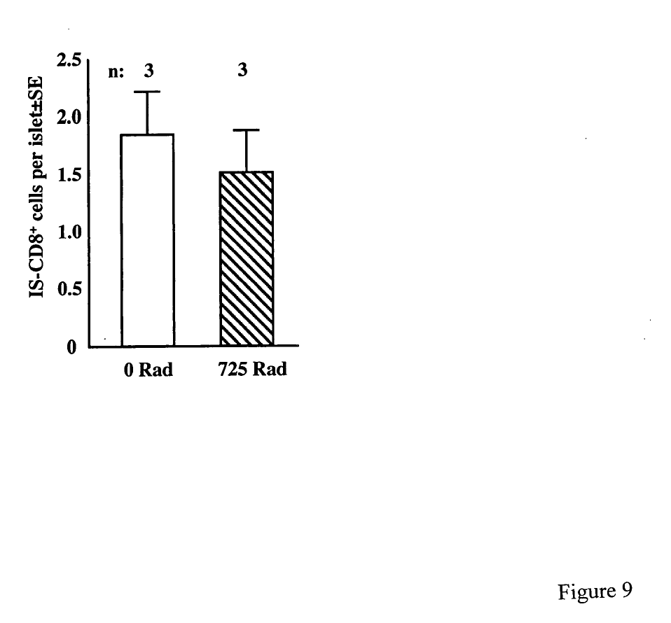

Since we routinely irradiate recipient mice to create a “niche” for donor cells and to minimize host response to incompatible donor cells, we tested whether irradiation affects homing to the islets. There was no difference in homing of IS-CD8⁺ cells into the islets of irradiated and non-irradiated NOD.Rag1-KO mice (FIG. 9). Hence, irradiation was not responsible for the “activation” of endothelium in inflammation-free animals. Thus, IS-CD8⁺ cells can home to the islets that do not have prior inflammation and their homing is quite tissue-specific. IS-CD8⁺ cells express cellular adhesion molecules CD31, CD44, and CD54; integrins CD11a (αL chain), CD103 (αIEL chain), CD49b (α2 chain), CD49d and CD29 (α4 integrin VLA-4), andCD18 (β2 integrin LFA-1), as well as P-selectin ligands (not shown). L-selectin, LPAM, E-cadherin and CD49a molecules were absent from IS-CD8⁺ cells' surface (not shown). Low levels of ligands for LFA-1 are known to be constitutively expressed by the endothelium of the islets, while the VLA-4 ligand VCAM-1 is inducible (21-23). Thus, integrins and/or P-selectin ligands are likely to contribute to the early stages of adhesion to the pancreatic endothelium engaging constitutively expressed counterparts present in the absence of inflammation.

Homing to the Islets Requires MHC Class I Expression.

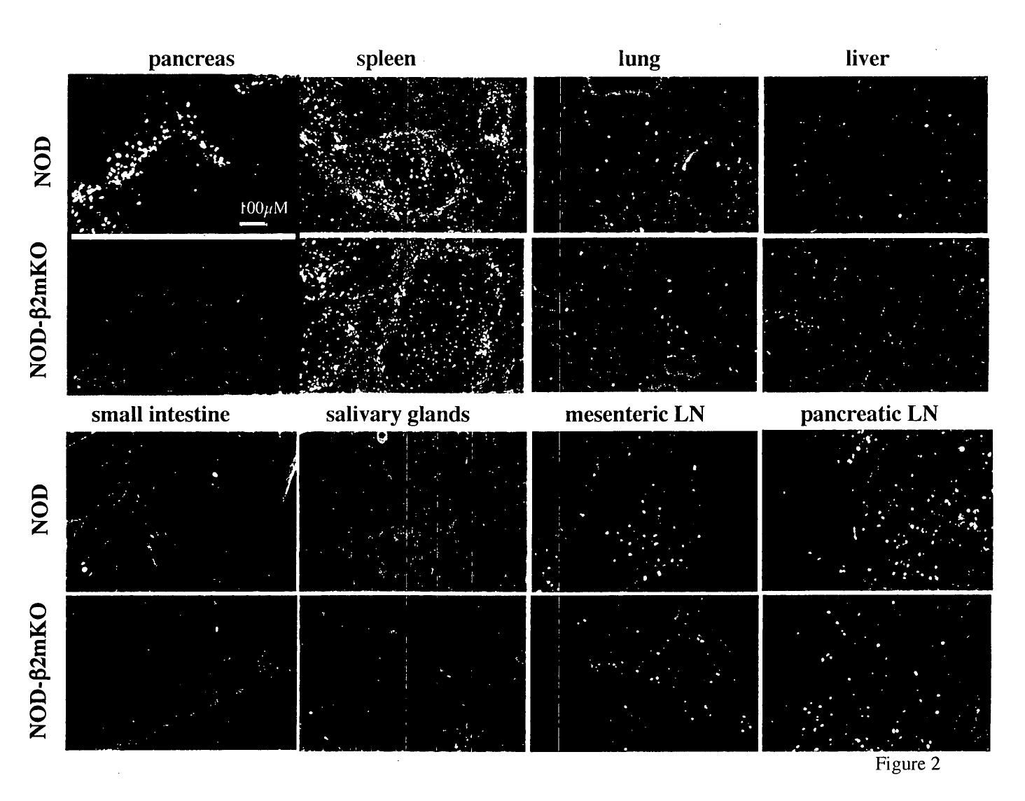

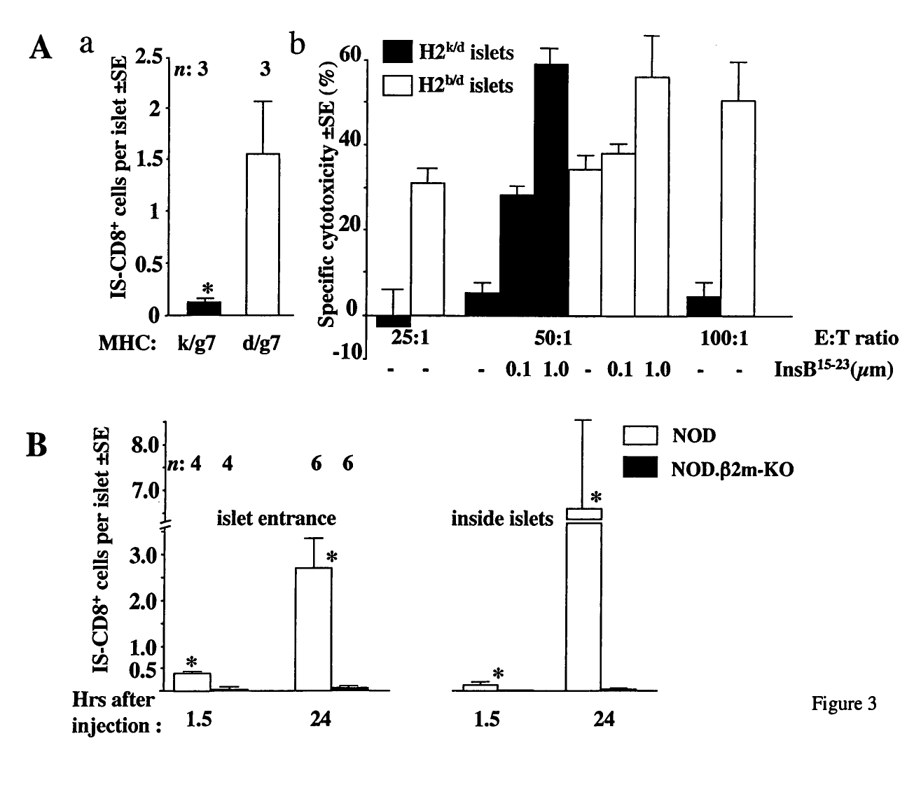

Since inflammation was not necessary for the initial steps in pancreatic homing of IS-CD8⁺ T cells, we tested whether homing of IS-CD8⁺ T cells could be antigen-driven. For that, NOD mice lacking MHC class I molecules (NOD.β2m-KO mice), were injected with labeled IS-CD8⁺ cells and their pancreata examined (FIG. 1D). There was hardly any significant number of IS-CD8⁺ cells in the isolated islets 24 hrs after injection. To show that pancreas was the only organ in the NOD.β2m-KO mice where IS-CD8⁺ cells refused to home, compared with NOD mice, we analyzed cryostat sections of several tissues from NOD and NOD.β2m-KO mice injected with Dil-labeled IS-CD8⁺ cells (FIG. 2). This systematic analysis of tissue distribution of the IS-CD8⁺ cells revealed that the difference between the two recipients was obvious only in the pancreatic islets. Evidence for peptide specificity came from the analysis of mice that expressed the H2^{k} MHC haplotype along with expression of the H2-K^{d} molecule. Such mice are resistant to IS-CD8⁺ cell-induced diabetes (see Table 1 below), and resistance was linked to the lack of specific homing of activated IS-CD8⁺ cells to the islets in H-2^{k/g7} animals (FIG. 3A,a): 24 hrs after IS-CD8⁺ cell injection H-2^{k}-positive islets contained approximately 16-fold fewer labeled cells.

TABLE 1
IS-CD8⁺ cells fail to transfer diabetes into mice bearing
the H2^{k} allele.

| | | n | Mean day |
|---|---|---|---|
| Strain | H2 | diabetic/tested | of onset |
| C3D2 | k/d | 0/7 | |
| (C3D2 × NOD)F1^{a} | d/g7 | 8/10 | 5.8 |
| (C3D2 × NOD)F1^{a} | k/g7 | 0/8 | |
| (B10.BR × NOD)F1 | k/g7 | 0/5 | |
| (NOD.B10Sn-H2^{b} × NoD)F1 | b/g7 | 3/3 | 13.0 |
| (NOD.NON-H2^{nb1} × NOD)F1 | nb1/g7 | 11/13 | 7.6 |
| ^{a}A cross between C3H/HeJ (H2^{k}) and DBA/2J(H2^{d}) strains (called C3D2) expresses k and d alleles of MHC. When C3D2 mice were crossed to NOD mice (with H2^{g7} MHC haplotype with class I alleles K^{d} and D^{b}), the progeny that expressed H2^{k/g7} were resistant | | | |
| # to diabetes, while H2^{d/g7} positive animals were sensitive. Obviously, resistance to IS-CD8⁺ cells was linked to H2^{k}. Recipient mice were irradiated with 725 Rad and injected with 10⁷ IS-CD8⁺ T cells 24 hrs later. Diabetes was detected by measuring urine | | | |
| # glucose levels and confirmed by blood glucose measurement. Resistance of H-2^{k}- positive mice was not due simply to lower expression of K^{d} molecules, because control F1 mice that expressed H2^{b/g7} or H2^{nb1/g7} were susceptible to IS-CD8⁺ cells, while the amount | | | |
| # of K^{d} expressed was “diluted” by the same factor of two. This resistance was also not due to preferential elimination or functional inactivation of IS-CD8⁺ cells in H2^{k} × H2^{g7} mice: we injected IS-CD8⁺ cells into (C3D2 × NOD) F1 mice | | | |
| # with either H2^{k/g7} or H2^{d/g7} haplotype and isolated these cells from the spleens of mice on the day when H2^{d/g7} mice became diabetic. Recovered cells were then activated with specific peptide in vitro and tested for their ability to destroy peptide-positive targets. We found | | | |
| # no loss of activity in T cells that “went through” H2^{k/g7} mice, and these cells were also diabetogenic when injected into NOD recipients (not shown). | | | |

Further analysis revealed that the presence of H2^{k} (or closely linked genes) caused the lack of presentation of InsB¹⁵⁻²³ peptide by K^{d} molecules. This was shown by testing of the direct recognition of islet β cells from H2^{k/d} animals by IS-CD8⁺ cells. Islets from C3D2 (H2^{k/d}) and control B6D2 (H2^{b/d}) mice were dispersed to single cell suspensions, labeled with ⁵¹Cr, and exposed to IS-CD8⁺ cells. β cells from mice positive for H2^{k} were resistant to lysis (FIG. 3A,b). However, when the InsB¹⁵⁻²³ synthetic peptide was added exogenously, lysis was restored (FIG. 3A,b), arguing that the block of lysis was neither due to a general malfunction of K^{d} molecules, nor to activation of inhibitory receptors on the CTL. The only reasonable explanation for this finding was that products of genes linked to H2^{k} were prohibiting the formation of functional K^{d}/insB¹⁵⁻²³ complexes. Thus, the absence of specific antigenic complexes, and not the general deficiency in MHC class I (found in NOD.β2m-KO mice), is critical for the lack of specific homing of IS-CD8⁺ cells to the pancreas.

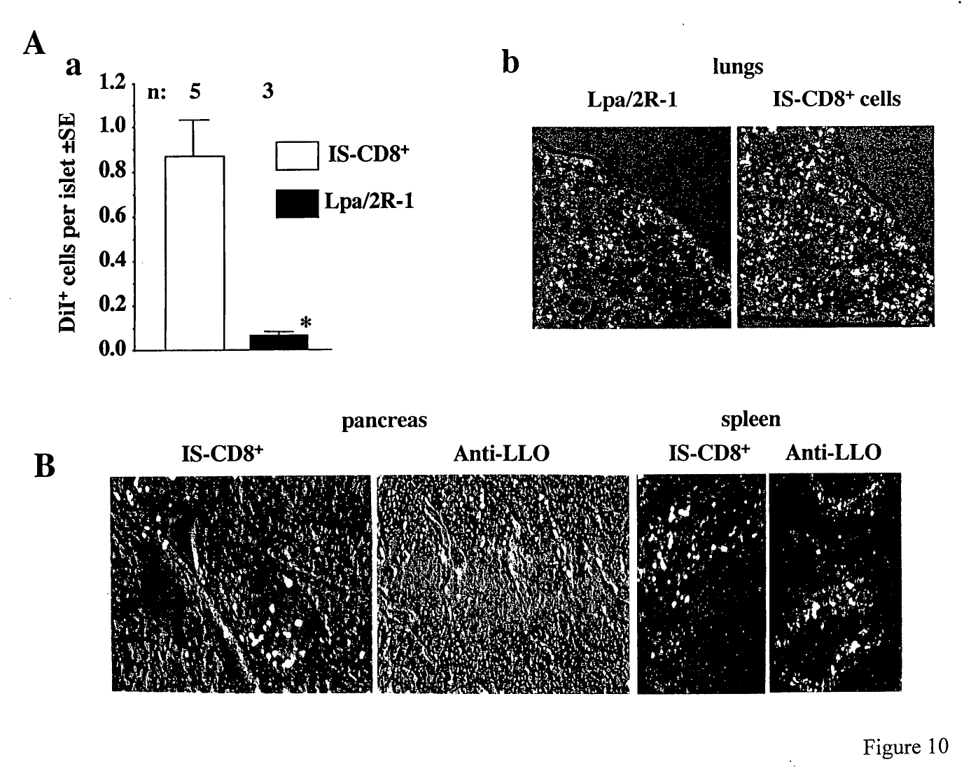

To confirm that homing to the islets is not a property of any activated T cell, we also used T cell clones with different specificities. DiI-labeled cells of LPa/2R-1 CD8⁺ clone (13) reactive to D^{b} complex with a peptide from minor histocompatibility antigen H3a^{a} which is widely expressed in NOD mice were not detectable in the pancreatic islets 2 or 24 hrs after injection, while the numbers of the LPa/2R-1 cells in the other tissues such as lungs (FIG. 10) were comparable to the numbers of IS-CD8⁺ cells. Similarly, two other CD8⁺ T cell lines, D^{b}-restricted H3a^{b}-specific clone B/L, (recognizing H3a^{b} allele not expressed in NOD mice) (13) (not shown), and L12.3, K^{d}-restricted anti-listeriolysin clone (14) migrated to spleens but not to pancreata of NOD recipients (FIG. 10).

Thus, homing of IS-CD8⁺ cells appears to be both antigen-driven and pancreas-specific. However, IS-CD8⁺ cells need to cross the endothelial cell layer in order to penetrate the islets. Taking in consideration that homing also occurs in the absence of local inflammation, we hypothesized that endothelial cells present MHC-insulin peptide complexes to T cells, stimulating their adhesion and penetration into the islets.

Endothelial Cells from Pancreatic Islets can be Recognized by IS-CD8⁺ Cells in vivo and in vitro.

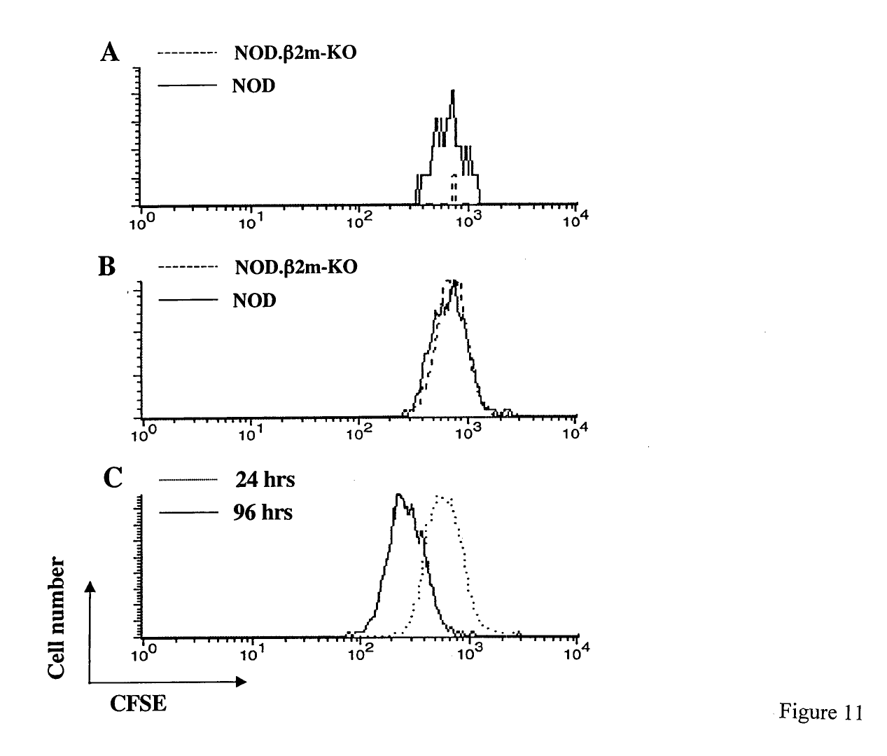

To test this hypothesis, we further used NOD.β2m-KO mice as recipients of DiI-labeled IS-CD8⁺ T cells. Their islets were analyzed at different time points after injection of IS-CD8⁺ cells (FIG. 3B). Morphometric analysis allows not only a calculation of the numbers of infiltrating cells, but also reveals their localization. The numbers of cells accumulated in different locations reflect the process of T cell homing. As expected the numbers of IS-CD8⁺ cells found in the MHC class I-deficient islets 24 hours after injection were extremely small compared to those found in the islets of MHC-sufficient NOD mice. Most importantly, very few T cells were found to be located at the islet vascular isthmus in MHC class I-negative NOD mice. If their firm adhesion to the endothelium was MHC class I-independent, we would be able to detect IS-CD8⁺ cells in the vascular isthmus of NOD.β2m-KO mice, which was not the case. It was possible, however, that accumulation of IS-CD8⁺ cells in the islets of NOD mice was exclusively due to a secondary activation of endothelium caused by recognition of K^{d}-insulin peptide complexes in the islets themselves, which would be impossible in β2m-negative mice. That would lead to fast “passing-through” of IS-CD8⁺ cells in β2m-negative islets, and hrs after injection we would be simply unable to detect that. However, we were able to detect IS-CD8⁺ cell accumulation at the entrance rather early (1.5 hr after injection) in NOD mice (FIG. 3B). Importantly, such accumulation preceded penetration into the islets. In contrast, at no time points could we detect accumulation of IS-CD8⁺ cells in β2m-negative mice at the islet isthmus or inside the islets. These results argue that not only the islet penetration, but also the adhesion to endothelium, were abolished by elimination of MHC class I expression. Moreover, accumulation with time of IS-CD8⁺ cells at the isthmi and inside the islets is not due to local proliferation, but due to additional recruitment from the bloodstream. Twenty four hrs after injection we found no explicit proliferation of CFSE-labeled IS-CD8⁺ cells isolated from the islets compared to IS-CD8⁺ cells isolated from the spleens (FIG. 11).

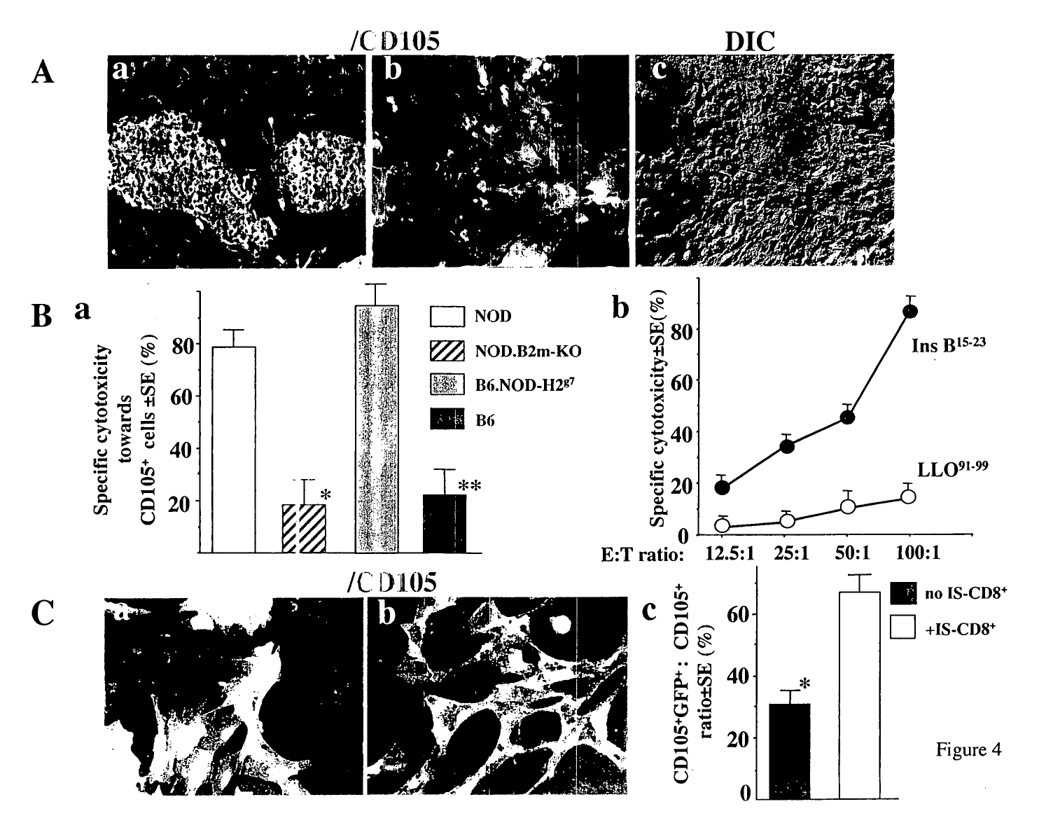

To further support the hypothesis that endothelial cells can function as APC for IS-CD8⁺ cells, we proceeded to show that endothelial cells in the pancreatic islets could be directly recognized by IS-CD8⁺ cells. For that we used islet organ cultures. On day 7 of the islet organ culture IS-CD8⁺ cells were added for 12 hrs. Immediately after that, the live cultures were stained with antibodies against a β cell marker, glucose transporter 2 (Glut-2), and an endothelial marker CD105 (endoglin). Glut-2⁺ and CD105⁺ cells are clearly non-overlapping populations in islets of the intact pancreas or in isolated islets in culture (FIG. 4A). The numbers of remaining CD105⁺ cells were counted in islet cultures exposed or not to IS-CD8⁺ cells. In the cultures of MHC class I-sufficient NOD or B6.NOD-H2^{g7} (congenic strain with H2^{g7} on B6 background) islets the majority of the Glut-2⁺ cells were destroyed by IS-CD8⁺ cells. Importantly, most of the CD105+ cells were also destroyed. In NOD.β2m-KO or B6 islet cultures, on the other hand, Glut-2⁺ and CD105⁺ cells remained intact, indicating that the killing of CD105⁺ cells (or CD31⁺ cells in some experiments, not shown) was specific and that B6 genetic background did not matter for susceptibility to specific killing (FIG. 4B,a). Moreover, when NOD endothelial cells were isolated from non-islet source (aorta) and cultured without insulin-producing β cells, they were insensitive to IS-CD8⁺ cells cytotoxicity unless exogenous Ins-B¹⁵⁻²³ peptide was added (FIG. 4B,b). However, the possibility existed that in NOD islet cultures the recognition by IS-CD8⁺ cells of their primary targets, the antigen-expressing β cells, led to secretion of cytotoxic cytokines that killed endothelial (CD105⁺) cells non-specifically. This would not have occurred in NOD.β2m-KO or B6 islet cultures, as MHC class I K^{d} molecules were not present, or in aortal endothelial cells. To address this caveat, we cultured a mixture of the islets isolated from NOD and B6-GFP mice, which ubiquitously express green fluorescent protein (GFP). These mixed islet cultures were exposed to IS-CD8⁺ cells and stained with anti-CD105 (FIG. 4C). The ratio of cells double-expressing GFP and CD105⁺ to the total number of CD105⁺ cells was measured by direct cell counting using fluorescent microscopy. The results revealed that NOD-derived GFP⁻CD105⁺ cells were eliminated, while B6-derived GFP⁺ CD105⁺ cells remained intact (FIG. 4C,c). That suggested that CD105⁺ cells must express the cognate MHC-peptide complex to be destroyed by IS-CD8⁺ cells.

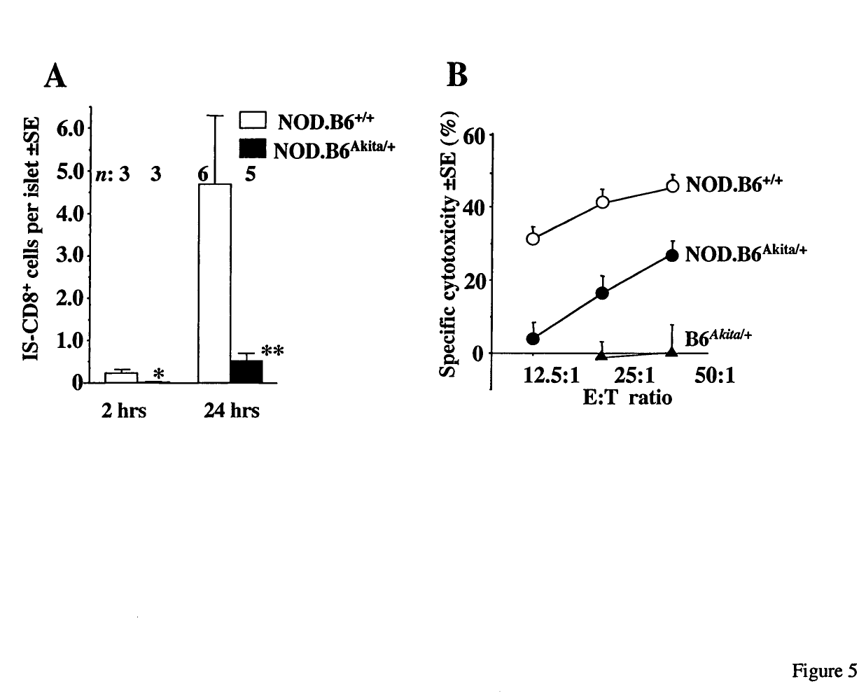

How is the insulin peptide-K^{d} complex acquired by endothelium? High local concentration of insulin suggests that secreted protein may be taken up by endothelial cells and then cleaved to peptides. If that was true, β cells that make insulin (and insulin peptide-K^{d} complexes) but have impaired insulin secretion, would be susceptible to lysis by CTL but inaccessible for them because the endothelium is not cross-presenting the antigen. Support for this hypothesis came from the analysis of IS-CD8⁺ cells trafficking into the islets of NOD.B6^{Akita/+} mice homozygous for H2^{g7} and heterozygous for the dominant mutation designated Ins2^{Akita} in the insulin 2 gene. This point mutation (Cys96Tyr) affects processing and secretion of both insulin I and insulin 2 proteins in a dominant fashion, so that insulin release is greatly diminished and diabetes develops very early (24, 25). Labeled IS-CD8⁺ cells were found to home significantly less efficiently (tested at 2 and 24 hrs after injection) to the islets of mice bearing the Ins2^{Akita} mutation than to the islets of their wild-type littermates (FIG. 5A). Very importantly, β cells isolated from mutant mice were sensitive to IS-CD8⁺ cells in vitro (FIG. 5B). Because of the substantial depletion of β cells from the pancreatic islets of Ins2^{Akita} mice (24), the level of cytotoxicity for mutant islets was lower than that observed for wild-type islets (FIG. 5B). Nevertheless, IS-CD8⁺ cells were clearly capable of direct recognition of the islet cells of NOD.B6^{Akita/+} mice. Thus, IS-CD8⁺ cells were able to recognize β cells but stopped short of adhesion to the islet vasculature. This argues that secretion of insulin, the protein donor of antigenic peptide, is essential for presentation of the K^{d}-Ins¹⁵⁻²³ complex by endothelial cells, and suggests that generation of MHC-insulin peptide complexes depends on degradation of insulin by endothelial cells.

Chemokine Receptors are Involved in IS-CD8⁺ Cell Homing.

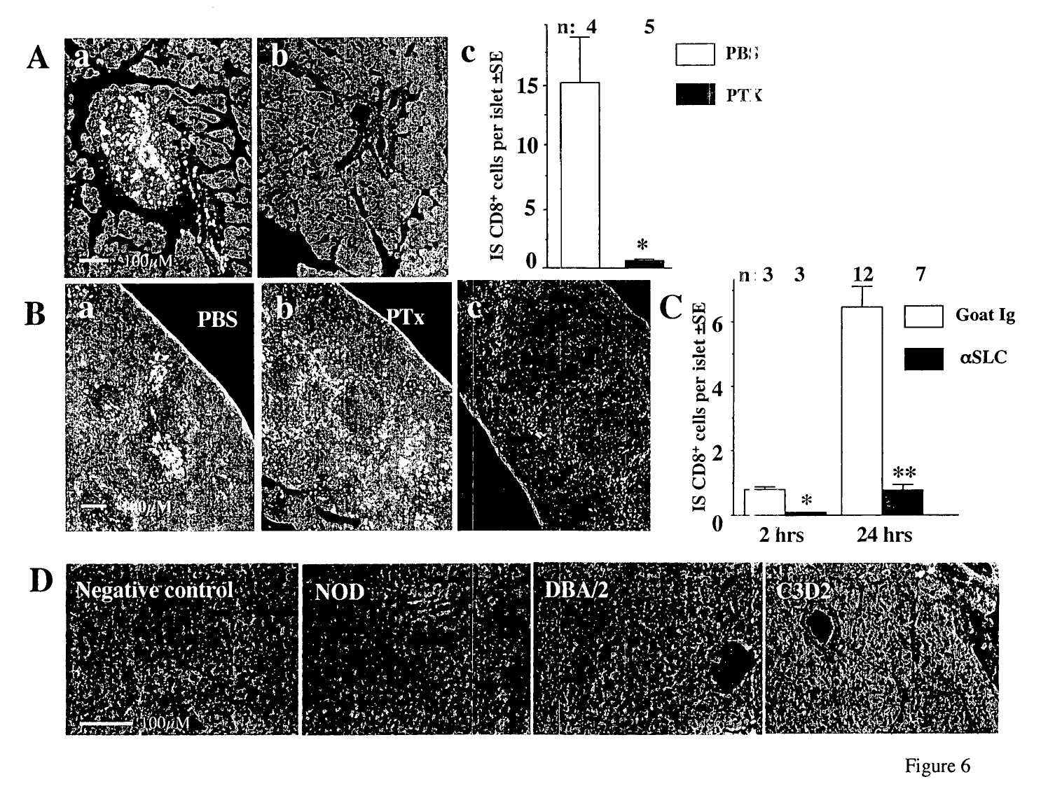

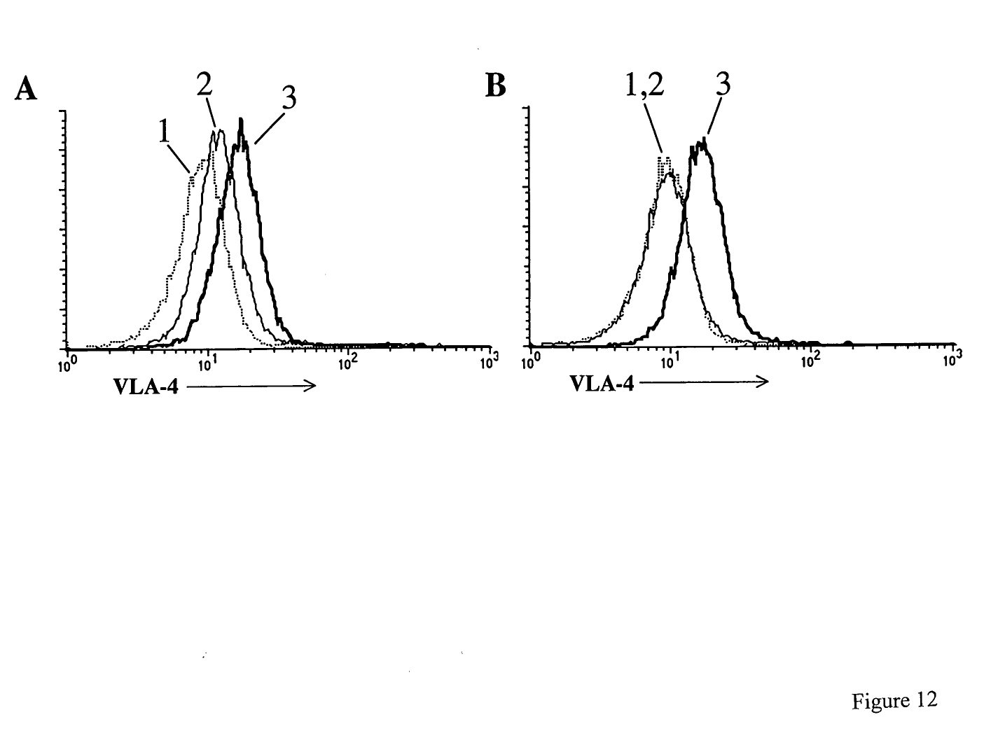

Although signaling through TCR leads to activation of integrins on the T cell surface (26-28) (we found that it was also true for the particular IS-CD8⁺ cell clone that we were using, see FIG. 12), it is unlikely that this mechanism alone could account for the strength of interactions with the endothelium needed for complete arrest of T cell rolling. Integrin activation may be also triggered through G-protein-coupled chemokine receptors (29-32). Since homing of lymphocytes usually depends heavily on the recognition of tissue-specific chemokines (33), we sought to address the question of whether chemokine receptors are involved in trafficking of IS-CD8⁺ cells to the pancreatic islets. We started with treatment of IS-CD8⁺ cells with pertussis toxin (PTx), a potent inhibitor of G-protein-coupled receptor signaling. IS-CD8⁺ cells treated with PTx or PBS were stained with DiI and injected into NOD mice. The cells treated with PTx did not lose their viability and responded normally to InsB¹⁵⁻²³ peptide stimulation 24 hrs after treatment (not shown). The presence of IS-CD8⁺ cells in the pancreatic islets was assessed 24 hrs after injection by analysis of cryostat sections (FIG. 6A). PTx-treated IS-CD8⁺ cells were practically absent from the islets, suggesting the possibility that chemokine receptors were involved in the homing of IS-CD8⁺ cells. The multitude of chemokines and their receptors complicates the task of identifying of a specific pair. However, when we examined lymphoid organs of NOD mice injected with PTx-treated IS-CD8⁺ cells, we detected a clue to the identity of the chemokine that could assist in trafficking to the pancreatic islets. In our experiments, the presence of labeled IS-CD8⁺ cells in the spleen always serves as an internal control for the quality of the transfer. When NOD mice were injected with IS-CD8⁺ cells treated with PTx, we found that the distribution of these cells in the spleen was different from the distribution of untreated cells: PTx-treated cells avoided T cell zones (FIG. 6B,a,b). Homing of T cells to the T cell zones of the secondary lymphoid organs is controlled by a pair of chemokines, CCL21 (also known as 6Ckine, Exodus-2, SLC) and CCL19 (MIP-30, Exodus-3, ELC) (34, 35). SLC expression in the T cell zones of spleen by immunofluorescence is shown in FIG. 6B,c. Since both SLC (CCL21) and ELC (CCL19) chemokines have been shown to trigger β2 integrin affinity and mobility changes promoting LFA-1-mediated lymphocyte adhesion (36, 37), we asked whether these chemokines were involved in the homing of IS-CD8⁺ cells to the pancreatic islets.

First, we blocked SLC function by treating mice with anti-SLC antibodies in vivo, followed by the transfer of labeled IS-CD8⁺ T cells. Cryostat sections of the pancreata from mice injected with anti-SLC were compared to those from mice injected with normal goat Ig by counting labeled cells per islet (FIG. 6C). Anti-SLC antibodies had a profound effect on the IS-CD8⁺ T cells' ability to adhere to the islet endothelium (2 hrs and 24 hrs after injection) and to penetrate the islets (24 hrs after injection). Clearly, the homing of IS-CD8⁺ T cells decreases if SLC is neutralized. The antibodies did not cause any loss of injected cells as their numbers in peripheral blood and spleens were similar (not shown).

Second, we stained cryostat sections of the pancreata from different mice with anti-chemokine antibodies. We found that SLC was expressed in the isthmus of the islets (FIG. 6D). Most of the SLC-positive cells are localized to lymphatics and lack endothelial markers (not shown), but SLC is easily and highly diffusible: intradermal injection of SLC affected homing of T cells to the regional lymph nodes (38), and ectopic over-expression in β cells (39, 40) recruited lymphocytes to the islets. Importantly, SLC expression was found in the islets of diabetes-prone NOD mice, as well as in the islets of mice with no spontaneous diabetes and no pre-existent inflammation, i.e., DBA/2J (H2^{d}) and C3D2 (H2^{kxd}) mice. Of the latter two strains, DBA/2J mice were sensitive to the transfer of IS-CD8⁺ T cells, while C3D2 mice were resistant.

Two important conclusions can be drawn from these findings: a) chemokines, namely SLC, play a critical role in IS-CD8⁺ T cells homing to the islets of Langerhans; b) SLC expression does not correlate with inflammation and is not sufficient per se for the homing of IS-CD8⁺ T cells, and is likely to act in concert with recognition of an islet-specific peptide presented by endothelial cells.

Triggering of TCR and Chemokine Receptors of IS-CD8⁺ Cells Cooperate in Strengthening T Cell Adhesion to the Endothelium.

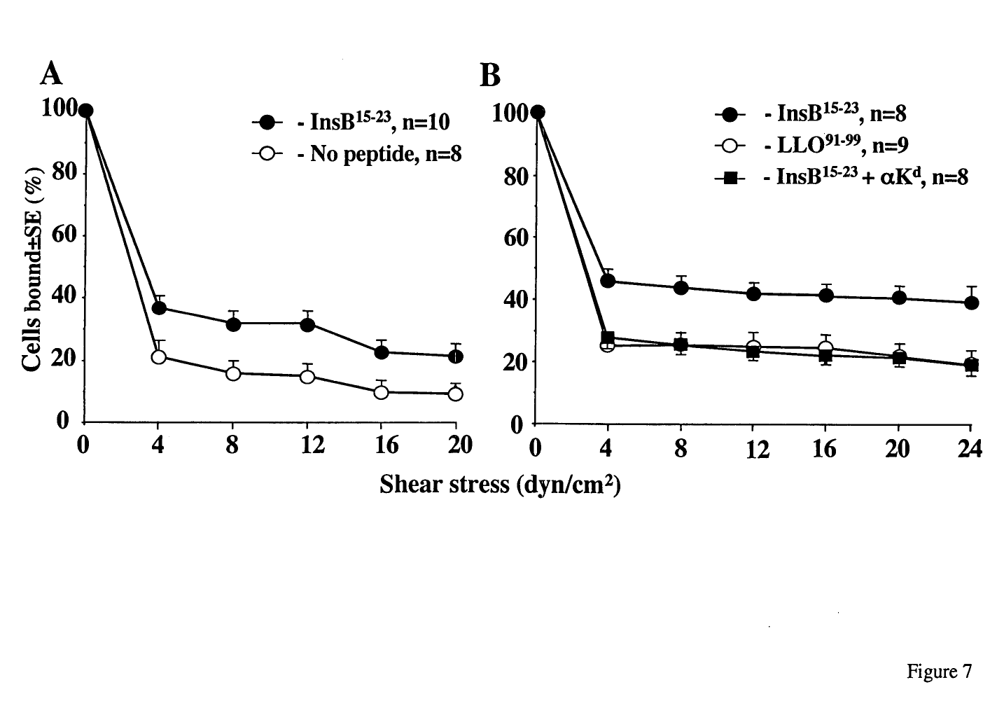

To test whether activation of IS-CD8⁺ cells through their TCR would lead to a detectable activation of integrins on their surface, we stimulated IS-CD8⁺ cells with anti-CD3 antibodies and immediately stained them with FITC-labeled fibronectin. Anti-CD3 stimulation led to increased binding of FBN-FITC to IS-CD8⁺ cells (FIG. 12A). FBN binds to α4 integrins with high affinity when they are activated (41-43). This binding, along with the low binding seen in IS-CD8⁺ cells prior to anti-CD3 exposure, was completely blocked by antibodies to VLA-4 integrin (FIG. 12B), but not by antibodies to LFA-1, which does not bind FBN. Thus, activation of T cells through their antigen-specific receptors led to the activation of integrins. These results clearly support the idea that recognition of specific MHC-peptide complexes may participate in the homing of IS-CD8⁺ cells, enhancing adhesion between T cells and endothelium. We applied an “adhesion under shear stress” approach to gain further support for our hypothesis that MHC-peptide recognition is important for T cell adhesion to endothelial cells and, hence, for homing. For that, the monolayers of aortal endothelium from NOD mice were used as targets for adherence of IS-CD8⁺ cells in the Glycotech flow chamber. T cells were allowed to adhere to the endothelial cells, and then the flow of tissue culture medium with several increments of pressure was initiated. It became clear that pre-cultivation of endothelial monolayers with the InsB¹⁵⁻²³ peptide (but not without the peptide or with an irrelevant K^{d}-binding peptide) led to increased adhesion of the T cells (FIG. 7A). However, to achieve adhesion significantly different from that in the no-peptide control, T cells had to be allowed to stay in contact with endothelial cells for 300 seconds prior to the start of flow. The adhesion, however, became stronger when T cells were pre-treated with SLC before the test: the time necessary for adhesion was reduced to 90 seconds, the fraction of adherent cells was larger, and more of them stayed attached longer than T cells that were not exposed to SLC. This adhesion was specific, as it occurred only in the presence of specific peptide, and it was possible to suppress it by addition of antibodies against H2-K^{d} molecules to the monolayers before the test (FIG. 7B). The pre-treatment with SLC also increased the baseline adhesion in the absence of specific peptide, but the cooperation of peptide and SLC made the specific adhesion stronger.

Thus, cooperation of multiple mechanisms regulating T cell adhesion to the endothelium is required for their specific homing. The two mechanisms described above are both complementary and essential for this process.

### Discussion

Homing of activated T cells to sites where they are most needed (where pathogens are present) or where they are most harmful (in autoimmunity) are likely to be controlled by similar mechanisms. Using T cells that recognize an antigen uniquely produced by a specialized tissue—the islets of Langerhans in the pancreas—we were able to analyze the interactions required for the islet-specific homing of CD8⁺ T cells. Homing is a multi-step process that allows T cells to cross into tissues through the endothelium of microcapillary vessels. Each of these steps (initial tethering, slow rolling, activation-dependent arrest, and diapedesis) is controlled by different interactions of surface molecules and multiple signaling pathways triggered by such interactions (30, 33).

Some embodiments of T cell trafficking to the pancreas, such as the role of integrins and their ligands, have been addressed previously. It has been shown that LFA-1/ICAM-1, VLA-1/VCAM-1, and α4 integrin/MadCAM interactions are important for the homing of diabetogenic T cells to the pancreas (21, 44, 45). It is widely believed (and rightly so) that inflammation that induces many adhesion molecules facilitates the homing of T cells. However, when we introduced IS-CD8⁺ T cells into mice with no pre-existent inflammation, the mice rapidly developed diabetes, and labeled IS-CD8⁺ cells were found in their islets (FIG. 1). This was taken as evidence that no inducible molecules are required for the initiation of homing of IS-CD8⁺ to the islets. Interestingly, IS-CD8⁺ cells were never found in salivary glands of NOD mice (FIG. 2), although NOD mice typically develop inflammation of salivary glands. Thus, inflammation by itself cannot determine the homing of IS-CD8⁺ T cells.

To explain the inflammation-independent organ-specific homing of IS-CD8⁺ cells, we put forward a hypothesis that endothelium can cross-present the antigen and thus participate in T cell adhesion in an antigen-specific fashion. We found experimental evidence to support this hypothesis: firstly, homing to the pancreas but not to other organs depended on expression of MHC class I and of the specific peptide (FIGS. 2, 3); secondly, IS-CD8⁺ cells up-regulated integrin avidity upon stimulation of their TCR (FIG. 12) and demonstrated adhesion to endothelial cells under shear stress in the presence of specific peptide (FIG. 7); thirdly, endothelial cells from pancreatic islets could be directly recognized in in vitro cytotoxicity assay (FIG. 4). This direct recognition did not occur due to the presence of insulin in the tissue culture medium, since aortal endothelium cultivated in the same medium was not recognized by IS-CD8⁺ cells (FIG. 4B,b). Thus, pancreatic endothelial cells have a necessary machinery to acquire insulin from β cells and produce the cognate peptide. The mode of acquisition of the K^{d}-InsB¹⁵⁻²³ complexes by endothelial cells is not yet established. However, the observation that IS-CD8⁺ cells have a reduced ability to home into the islets of NOD.B6^{Akita/+} mice (FIG. 5) suggested that the peptide was generated by endothelial cells from secreted insulin rather than acquired as a peptide or an MHC-peptide complex produced by β cells. It is possible, however, that in NOD.B6^{Akita/+} mice other properties of endothelium are affected by insulin deficiency reducing T cell adhesion.

The need in secreted insulin for peptide presentation suggests that the peptide is generated by endosomal rather than proteosomal degradation. This scenario is not unprecedented, as evidence in favor of alternatives to the conventional MHC class I peptide processing and presentation pathway is accumulating (46-48). These studies have shown that MHC class I molecules could be found in classical MHC class II compartments, and that the TAP-independent and proteosome-independent pathways can lead to presentation of peptides by MHC class I. The fact that endothelial cells can serve as APC has been established previously (10, 11, 49). Hepatic endothelial cells were found capable of presenting complexes of MHC class I molecules with peptides derived from an injected foreign soluble protein (50). However, cross-presentation of peptides derived from endogenously produced protein has not been described. Local concentration of insulin is very high amounting to 7% of total islet protein (51). Insulin produced locally in such high concentrations may be broken down into peptides by endothelial cells. We do not know yet whether endothelium is destroyed by IS-CD8⁺ cells in vivo, or it is activated to facilitate T cell adhesion and diapedesis. This is an important issue and a matter of future studies. Our own data (FIG. 3B) showed that secondary activation of endothelium is occurring with time, leading to an increase of the numbers of IS-CD8⁺ cells found in the isthmus.

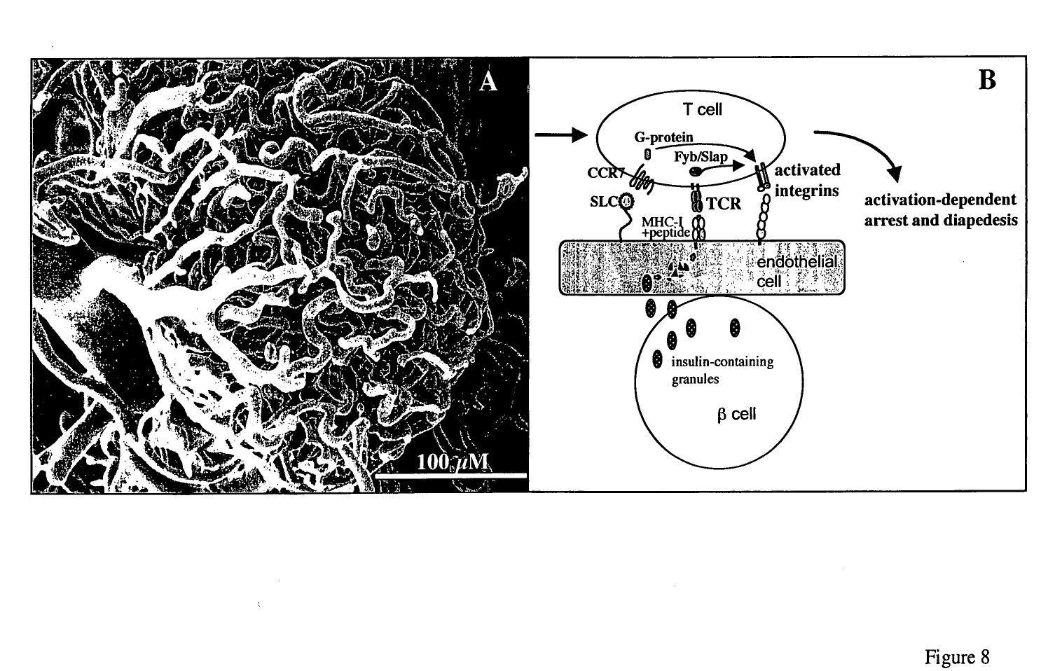

Firm adhesion of IS-CD8⁺ cells is also controlled by G-protein-coupled chemokine receptors. We have found that SLC is the chemokine responsible for homing of IS-CD8⁺ T cells (FIG. 6C). Its expression was detected in the pancreas by immunostaining (FIG. 6D), but it was present not only in NOD mice, which may have an ongoing inflammation, but also in DBA/2J mice (with no pre-existent inflammation) and in C3D2 mice, in which IS-CD8⁺ T cells do not home to the islets. It is very likely that at advanced stages of diabetes development, SLC expression in the pancreas of NOD mice increases enhancing the infiltration by T cells (52, 53). However, our data showed that constitutive presence of SLC in the pancreas in diabetes-free mice is required for IS-CD8⁺ homing, but by itself is not sufficient for successful homing. Similarly, in the in vitro adhesion under shear stress assay, SLC treatment of T cells slightly increased their adhesion to the endothelium without the cognate peptide. Nevertheless, the best adhesion was achieved when MHC/peptide complexes and SLC worked in concert (FIG. 7B). A model describing additive effects of antigen recognition and activation of the chemokine receptor CCR7 on the surface of IS-CD8⁺ cells is shown in FIG. 8. CCR7 expression by IS-CD8⁺ cells was confirmed by staining with ELC-Ig fusion protein, and by real-time RT-PCR (not shown). Thus, SLC-triggered integrin activation may be aided by TCR-mediated activation of integrins (26, 36, 41). We found that TCR-mediated integrin activation was also a property of IS-CD8⁺ cells, as the avidity of VLA-4 towards fibronectin increased after stimulation with anti-TCR antibody (FIG. 12). Other integrins (e.g., LFA-1) are likely to be activated in IS-CD8⁺ cells in a similar fashion after TCR engagement. Homing specificity is maintained by fulfillment of both requirements (presence of appropriate peptide and an appropriate chemokine receptor): a CCR7-positive T cell clone LPa/2R-1 with different specificity did not show any significant accumulation in the pancreatic islets 2 or 24 hrs after injection (FIG. 10). The results show that pancreatic endothelium is not randomly attracting any T cells that are capable of sensing SLC, but only those that have appropriate specificity (FIG. 8).

Our study suggests that endothelial cells may play a more prominent role in the trafficking of activated T cells than is currently appreciated, providing antigen-driven specificity of homing. It remains to be established how general this principle is. While insulin is a secreted protein, it is unclear whether similar mechanisms may work for non-secreted antigens. However, two recent studies (49, 54) suggest that endothelium (at least in transplantation models) can cross-present non-secreted antigens. It is difficult to underestimate the significance of such a mechanism for the development of autoimmune diabetes. Because IS-CD8⁺ cells were found to be a predominant fraction of the islet-infiltrating cell population in NOD mice very early in the pathogenesis of diabetes (5) they may provide assistance to other T cells in the penetration of islets through activation of the local endothelium. The presence of SLC and activating signals from IS-CD8⁺ cells may lead to the initial accumulation of lymphocytes at the vascular entrance (known as periinsulitis), which later develops into accumulation of lymphocytes within the islets (insulitis) and damage to the insulin-producing cells (diabetes).

### Materials and Methods

Mice—C57BL/6J (B6), C57BL/6-TgN(ACTbEGFP)1Osb (B6-GFP), NOD/LtJ (NOD), DBA/2J, BALB/cJ, (C3H/HeJxDBA/2J) F1 (C3D2), (C57BL/6JxDBA/2J) F1 (B6D2), NOD.129P2(B6)-B2m^{tm/Un}c/J (NOD.β2m-KO), NOD.129S7(B6)-Rag1^{tm/Mom} (NOD.Rag1-KO), NOD.B10Sn-H2^{b}, NOD.NON-H2^{nbl}, B6.NOD-D17Mit21-D17Mit10 (H2^{g7}, Idd1) (B6.NOD-H2^{g7}), and B10.BR-H2^{k} H2-T18^{a}/SgSnJ (B10.BR) mice were obtained from The Jackson Laboratory (Bar Harbor, Me.). C57BL/6-Ins2^{Akita} (B6^{Akita/+}) mice and B6^{Akita/+} backcrossed to NOD for N2-3 (NOD.B6^{Akita/+}) were a generous gift of Dr. Leonard Shultz (The Jackson Laboratory). All animals were housed in a specific pathogen-free research facility.

CD8⁺ T cell clones—Insulin-specific, K^{d}-restricted T cells, IS-CD8⁺ cells of the TGNFC8 clone (5) were maintained in vitro as previously described (6) in Click's medium (Irvine Scientific, Santa Ana, Calif.) supplemented with 5% fetal calf serum (FCS) (Sigma chemical Co., St. Louis, Mo.), 2×10⁻⁵ M β2-mercaptoethanol (Bio-Rad Laboratories, Hercules, Calif.), 20 mM penicillin-streptomycin mixture (Life Technologies, Rockville, Md.), 3 mg/ml L-glutamine (Life Technologies) and 5 U/ml mouse recombinant IL-2. Cells were stimulated every 3 weeks by irradiated (2000 Rad) NOD derived pancreatic islets, or by NOD splenocytes loaded with 10 μg/ml of the synthetic insulin B chain (InsB¹⁵⁻²³, LYLVCGERG) peptide produced by Research Genetics (Huntsville, Ala.). Pancreatic islets were isolated by a collagenase digestion method, and hand-picked in Hanks' solution (Life Technologies) after purification on a Histopaque 1119 (Sigma) gradient (12). Control D^{b}-restricted T cell clones LPa/2R-1 and B/L specific for the peptides derived from minor histocompatibility antigen H3a (alleles H3a^{a} and H3a^{b} respectively) (13) were kindly provided by Dr. Derry Roopenian (The Jackson Laboratory). K^{d}-restricted anti-listeriolysin peptide (LLO⁹¹⁻⁹⁹) CD8⁺ T cell clone L12.3 (14) was a kind gift from Dr. Eric Pamer (Memorial Sloan-Kettering Cancer Center, New York, N.Y.).

Induction of diabetes—IS-CD8⁺ cells were washed with PBS, counted, and injected i.v. at 10⁷ cells per animal into irradiated (725 Rad, 24 h. in advance) recipients. Both males and females 5-8 weeks of age were used. Diabetes was detected by daily monitoring for 21 days of glucose levels in urine, using Diastix reagent strips (Bayer Corp., Elkhart, Ind.).

Fluorescent tracing and morphometric analysis—For trafficking studies, IS-CD8⁺ and control T cells were incubated at 10⁷ cells /ml for 30 min at 37° C. in the dark in complete medium containing 5% FCS and 0.0075 mg/ml of fluorescent dye didodecyltetramethylindocarbocyanine perchlorate (DiI) (Molecular Probes, Eugene, Oreg.), then washed 3 times with PBS. For analysis of short-term proliferation and adhesion studies IS-CD8⁺ cells were stained in vitro with fluorescent dye carboxyfluorescein diacetate succinimidyl ester (CFSE) (Molecular Probes, Eugene, Oreg.) as described (12). 10⁷ of labeled IS-CD8⁺ cells were injected i.v. into irradiated (725 Rad, 24 h in advance) animals. Mice were sacrificed at time points indicated, and their spleens, pancreata, lymph nodes, peripheral blood and lungs were removed, lymphocytes were isolated as described (5), stained with allophycocyanin-conjugated anti-CD8 (53-6.7) antibody (BD Pharmingen), and subjected to FACS analysis. Alternatively, organs were fixed in 0.1 M periodate-lysine-paraformaldehyde phosphate buffer, sucrose-saturated, and freeze-molded in OCT compound (Sakura Finetek Inc., Torrance, Calif.). Seven-μm thick cryostat sections of the entire pancreas were obtained at 60 μm intervals using a Leica CM1900 cryotom, (Leica A G, Heerbrugg, Switzerland). Distribution of Dil-labeled CD8⁺ cells within the islets was examined using a fluorescent microscope DMLB (Leica A G). At least 100 islets per mouse were examined. Cells were counted within areas relevant to a given islet (see FIG. 1 for representative image). Each IS-CD8⁺ cell was ascribed a specific position: either at the islet entrance (attached to the capillary wall or located in the isthmus) or inside the islet.

Monoclonal antibodies and FACS analysis—IS-CD8⁺ cells were stained in FACS buffer (PBS with 1% FCS and 0.1% NaN3) with anti-CD11a (2D7), anti-CD18 (C71/16), anti-CD31 (MEC13.3), anti-CD44 (1M7.8.1), anti-CD49d (SG31), anti-CD62L (MEL-14), anti-CD54 (3.E.2), anti-CD103 (2-E7), anti-LPAM (DATK-32), mAbs (all BD Pharmingen, San Diego, Calif.); anti-CD29 mAb (Chemicon Corp., Irvine, Calif.); anti-CD49a (Ha 31/8), anti-CD49b (Ha 1/29) mAbs (Biogen Corp., Boston, Mass.), and anti-E-cadherin (DECMA-1) mAb (Sigma). This was followed by staining with appropriate FITC- or PE-conjugated secondary antibodies (BD Pharmingen). Staining with mouse P-selectin-human Ig fusion protein (BD Pharmingen), and mouse ELC-human Ig fusion protein (gift of Dr. Ulrich von Andrian, Harvard University, Boston Mass.) was followed by goat-anti-human Ig-FITC conjugate (Sigma). IS-C8⁺ cells were counterstained with PE or FITC-conjugated anti-CD8 antibody (Sigma). For genotyping of H2^{k/g7} and H2^{d/g7} segregants of (C3D2)F1×NOD origin, mouse PBL were stained with FITC-conjugated anti-H2-K^{k} (36.7.5 or 16-3-1) antibodies (BD Pharmingen), in combination with Red-316-conjugated anti-CD4 (BRL) and PE-conjugated anti-CD8 antibodies (BD Pharmingen). Multi-color analysis was performed using a FACScan flowcytometer and utilizing Cellquest software (Becton-Dickinson, Mountain View, Calif.).

Chromium release cytotoxicity assay—Pancreatic islets or monolayers of cultured aortal endothelial cells were dispersed into single-cell suspension by incubation in cell-dissociation buffer (Sigma), labeled with 100 μCi of Na₂⁵¹CrO₄ (ICN Pharmaceuticals, Costa Mesa, Calif.) in 200 μl of complete Click's medium containing 5% FCS for 2 h at 37° C., washed three times, and co-cultured for 8-12 hours in 96-well plates (10⁴ targets per well in 200 μl of Click's medium with 5% FCS) with effector IS CD8⁺ cells at different effector-to-target ratios with different concentrations of InsB¹⁵⁻²³ peptide. To confirm the cytotoxic potential of IS-CD8⁺ cells, labeled P815 mastocytoma (H2^{d})cells were loaded with different concentrations of InsB¹⁵⁻²³ peptide and used as targets in the 4 hrs cytotoxicity assay. Specific cytotoxicity was based on the measurement of ⁵¹Cr release in 100 μl aliquots of cell-free supernatant using a gamma-counter (Wallac, Turku, Finland) and calculated using the formula: Specific Cytotoxicity (%)=[(Experimental Release−Spontaneous Release)/(Maximum Release−Spontaneous Release)]×100%.

Immunohistochemistry—Cryostat sections of fresh-frozen pancreas were cut 7 μm thick, immediately fixed in cold acetone (Fisher Scientific, Pittsburgh, Pa.) (3 min at −20° C.), air-dried for 2 hours, and stained for 1 hr at room temperature with mAbs against adhesion molecules: anti-VCAM-1 (429), anti-MadCAM (MECA-367), anti-PECAM (MEC 13.3), anti-CD34 (RAM34), anti-PNAd (MECA-79), anti-ICAM-1 (3.E.2), anti ICAM-2 (3C4); endothelial cells marker: anti-CD105 (MJ7/18) (all BD Pharmingen), and polyclonal antibodies against the β cell marker Glut-2 (whole anti-Glut-2 rabbit serum kind gift from Dr. Bernard Thorens, University of Lausanne, Switzerland), and polyclonal goat anti-mouse antibodies against chemokine anti-SLC (R&D Systems, Minneapolis, Minn.). Staining with corresponding FITC- or Rhodamine-labeled secondary antibodies (Jackson Immunoresearch, West Grove, Pa.) followed. After the final wash, slides were mounted in Vectashield mounting medium (Vector Laboratories, Burlingame, Calif.) and examined using fluorescent microscopy.

Pancreatic islet cultures and cytotoxicity assays—Pancreatic islets were cultured in Click's medium supplemented with 10% FCS and 0.1 mg/ml of Endothelial Cell Growth Supplement (ICN Pharmaceuticals, Costa Mesa, Calif.) in 8-well chambered glass slides (Nalge Nunc, Naperville, Ill.), pre-coated with 2% gelatin (Sigma). Seven days later, IS-CD8⁺ cells were added to the half of the chambers containing islet cultures for 12 hours, while the other half was left unaltered. Live cultures were stained for 1 hr at room temperature with affinity-purified rabbit anti-Glut-2 polyclonal antibodies and rat anti-CD105 (MJ7/18) or anti-CD31 (MEC13.3) mAbs followed by two 5-min washes and addition of corresponding secondary antibodies. After the final wash, chambers were detached, and slides were mounted in Vectashield medium and immediately examined. CD105⁺ or CD31⁺ cells in each well were counted, and the following formula was used to measure specific cytotoxicity: Specific Cytotoxicity (%)=(mean # of CD105⁺ cells in intact wells—mean # of CD105⁺ cells in the wells exposed to IS-CD8⁺): [(mean # of CD105⁺ cells in intact chambers)]×100%. Mixed NOD and B6-GFP islet organ cultures were exposed to IS-CD8⁺ cells or left intact, stained for CD105, and after the final wash were fixed with 4% paraformaldehyde (Sigma) for 20 min at 4° C. Slides mounted in Vectashield medium were examined under the fluorescent microscope. The ratio of GFP⁺ CD105⁺ cells to CD105⁺ cells was determined for each well. At least 200 cells/well of 3-4 wells per experiment were counted.

Fibronectin binding assay—IS-CD8⁺ cells harvested four days after IL-2 stimulation were used for the soluble fibronectin (FBN) binding assay performed as described (15). Briefly, 5×10⁶ IS-CD8⁺ cells were preincubated with 5 μg/ml of biotinylated 145-2C11 (anti-CD3) antibody (BD Pharmingen) in Click's medium containing 5% FBS for 30 min on ice, washed two times in PBS buffer, and cross-linked with 5 μg/ml of avidin-PE conjugate (BD Pharmingen) in the presence of 100 μg/ml of FITC-labeled FBN or FITC-labeled BSA (Sigma) as a control. FITC-labeling was carried out using the Fluorotag FITC conjugation kit (Sigma) according to the manufacturer's instructions. Purified R1-2 (anti-VLA-4) and M17/4 (anti-LFA-1) antibodies were added at a concentration of 5 μg/ml during the addition of soluble FBN. Upon addition of labeled FBN-FITC or BSA-FITC, cells were incubated for 30 min on ice followed by transfer to a 37° C. water bath for 10 min. After two washes with ice-cold FACS buffer, cells were analyzed immediately by FACS.

Primary vascular endothelium cell culture—Isolation and culture of vascular endothelium from mouse aorta was performed as described (16). Briefly, neutralized collagen extracellular matrix was aliquoted into 24-well plates, allowed to gel at 37° C. for 60 min, and equilibrated overnight with complete endothelial cell medium consisting of complete Click's medium with 20% FCS, and 50 μg/ml of Endothelial Cell Growth Supplement (Becton-Dickinson). The segments of thoracic aorta from adult NOD mice were placed endothelial-side down onto the collagen gel. Thirty-six hours later, 1 ml of complete endothelial cell medium was carefully added. After 3-5 days, the collagen gel was digested with a 0.3% collagenase H (Sigma) solution and the released cells were transferred to a T25 tissue culture flask. After reaching confluence, cells were detached by incubation with cell-dissociation buffer (Sigma), and split at a 1:3 ratio. Passage 2 to 5 cells were used for the experiments.

Parallel Plate Flow Chamber and Adhesion Assay—Aortal endothelial cells were plated on 35 mm Costar tissue culture dishes and allowed to reach confluence. The cells were then treated for 2 -3 hrs with 100 μM of InsB¹⁵⁻²³ or LLO⁹¹⁻⁹⁹ peptides in complete endothelial cell medium. In some experiments anti-K^{d} (SF 1-1.1) blocking mAbs were added for 20 min before the adhesion assay. The adhesion of IS-CD8⁺ cells under shear stress was examined with a parallel plate flow chamber obtained from Glycotech (Rockville, Md.) following the manufacturer's instructions and as previously described (17-19). A flow chamber with a 5-mm-wide gasket was used. Negative flow pressure was generated and controlled with an automated syringe pump (Braintree Scientific, Quincy, Mass.). IS-CD8⁺ cells were labeled with CFSE as described above, pretreated in some experiments with 100 ng/ml of mouse recombinant SLC (R&D Systems) in Click's medium for 30 min, and injected at a concentration of 3×10⁶/ml into the flow chamber. After IS-CD8⁺ cells were allowed to settle on the plate for the indicated length of time, the flow rate was increased stepwise every 10 s in increments of 1.28 m/min (˜4 dynes/cm² shear stress) to a maximum of 7.7 ml/min (corresponding to ˜24 dynes/cm²). Lymphocyte adhesion was visualized with a Leica inverted fluorescent microscope and recorded using a Spot-RT digital camera (Diagnostic Instruments, Sterling Heights, Mich.). Images were converted into digital movies and analyzed using Metamorph software (Universal Imaging Corp., Downingtown, Pa.). Labeled IS-CD8⁺ cells were counted in frames separated by equal time intervals from the start of the stress. Adhesion of cells in each frame was determined as a fraction (%) of the initial number of cells in the frame preceding the start of flow.

Treatment with pertussis toxin (PTx)—For treatment with PTx, IS-CD8⁺ cells were harvested, resuspended at 10⁷ cells/ml in complete medium containing 100 ng/ml of PTx (Sigma) and incubated for 2 h at 37° C. The final 30 min of incubation were combined with DiI-labeling. Treated cells were washed and injected into irradiated hosts or cultured under standard conditions in vitro.

Treatment with anti-SLC—Irradiated (725 Rad, 24 hours in advance) NOD mice were injected i.v. with 30-50 μg of polyclonal anti-SLC antibody (R&D Systems) or control goat Ig. DiI-labeled IS-CD8⁺ cells were injected 1 hr later. Mice were sacrificed at times indicated, and morphometric analysis of the pancreata was performed.

Pancreatic corrosion casting—Corrosion casts of pancreatic microcapillaries were produced as described (20) with modifications. Briefly, mice were sacrificed by overdose of anesthesia, perfused with 250 ml of warm PBS containing 1 u/ml of heparin and green food dye to ensure perfusion quality. Resin was prepared before injection by mixing Mercox, methyl methacrylate (Polysciences Inc, Warrington Pa.) and Catalyst at a 4:1:0.2 ratio and injected with a 30 g needle into the abdominal aorta near the branching of the upper mesenteric artery. Mercox and Catalyst were purchased as a kit from Ladd Research Industries, Burlington, Vt. In 5-10 min, pancreata with polymerized resin were excised, placed in 54° C. water for 1-2 hrs, incubated overnight with 2 mg/ml of pronase (Roche Diagnostics, Berkeley Calif.) in 50 mM Tris buffer with 20 mM EDTA, 2% SDS, pH8.0, washed in H₂O and immersed in 50% KOH solution at 50° C. for 24 hrs. Casts washed with hot running water were dried, mounted, gold-coated, and examined by SEM.

### References

1. Tisch, R., and McDevitt, H. O. 1996. Insulin-dependent diabetes mellitus. Cell 85: 291-297.
2. Delovitch, T. L., and Singh, B. 1997. The nonobese diabetic mouse as a model of autoimmune diabetes: immune dysregulation gets the NOD. Immunity 7:727-738.
3. Wong, F. S., and Janeway, C. A. Jr. 1999. The role of CD4 vs. CD8 T cells in IDDM. J. Autoimmun. 13:290-295.
4. Serreze, D. V., Leiter, E. H., Christianson, G. J., Greiner, D., and Roopenian, D. C. 1994.
   Major Histocompatibility Complex Class I-deficient NOD-B2m Null Mice are Diabetes and Insulitis Resistant. Diabetes 43:505-509.
5. Wong, F. S., Karttunen, J., Dumont, C., Wen, L., Visintin, I., Pilip, I. M., Shastri, N., Pamer, E. G., and Janeway, C. A. Jr. 1999. Identification of a MHC class I-restricted autoantigen in type 1 diabetes by screening an organ-specific cDNA library. Nat. Med. 5:1026-1031.
6. Wong, F. S., Visintin, I., Wen, L., Flavell, R. A., and Janeway, C. A. Jr. 1996. CD8 T cell clones from young nonobese diabetic (NOD) islets can transfer rapid onset of diabetes in NOD mice in the absence of CD4 cells. J. Exp. Med. 183:67-76.
7. Campbell, D. J., Kim, C. H., and Butcher, E. C. 2001. Separable effector T cell populations specialized for B cell help or tissue inflammation. Nature Immunol. 2:876-881.
8. Sebastiani, S., Allavena, P., Albanesi, C., Nasorri, F., Bianchi, G., Traidl, C., Sozzani, S., Girolomoni, G., and Cavani, A. 2001. Chemokine receptor expression and function in CD4⁺ T lymphocytes with regulatory activity. J. Immunol. 166:996-1002.
9. Osborn, L. 1990. Leukocyte adhesion to endothelium in inflammation. Cell 62:3-6.
10. Marelli-Berg, F. M., Frasca, L., Weng, L., Lombardi, G., and Lechler, R. I. 1999. Antigen recognition influences transendothelial migration of CD4⁺ T cells. J. Immunol. 162:696-703.
11. Pober, J. S., Kluger, M. S., and Schechner, J. S. 2001. Human endothelial cell presentation of antigen and the homing of memory/effector T cells to skin. Ann. NY Acad. Sci. 941:12-25.
12. Savinov, A. Y., Wong, F. S., and Chervonsky, A. V. 2001. IFN-gamma affects homing of diabetogenic T cells. J. Immunol. 167:6637-6643.
13. Zuberi, A. R., Christianson, G. J., Mendoza, L M., Shastri, N., and Roopenian, D. C. 1998. Positional cloning and molecular characterization of an immunodominant cytotoxic determinant of the mouse H3 minor histocompatibility complex. Immunity 9:687-698.
14. Busch, D. H., Pilip, I., and Pamer, E. G. 1998. Evolution of a complex T cell receptor repertoire during primary and recall bacterial infection. J. Exp. Med. 188:61-70.
15. Woods, M. L., Cabanas, C., and Shimizu, Y. 2000. Activation-dependent changes in soluble fibronectin binding and expression of betal integrin activation epitopes in T cells: relationship to T cell adhesion and migration. Eur. J. Immunol. 30:38-49.
16. Kreisel, D., Krupnick, A. S., Szeto, W. Y., Popma, S. H., Sankaran, D., Krasinskas, A. M., Amin, K. M., and Rosengard, B. R. 2001. A simple method for culturing mouse vascular endothelium. J Immunol Methods. 254:31-45.
17. Daniels, M. A., Devine ,L., Miller, J. D., Moser, J. M., Lukacher, A. E., Altman, J. D., Kavathas, P., Hogquist, K. A., and Jameson S. C. 2001. CD8 binding to MHC class I molecules is influenced by T cell maturation and glycosylation. Immunity. 15:1051-61.
18. Walcheck B., Kahn J., Fisher J. M., Wang B. B., Fisk R. S., Payan D. G., Feehan C., Betageri R., Darlak K., Spatola A. F., and Kishimoto T. K. 1996. Neutrophil rolling altered by inhibition of L-selectin shedding in vitro. Nature. 380:720-723.
19. Walcheck B., Moore K. L., McEver R. P., and Kishimoto T. K. 1996.
   Neutrophilneutrophil interactions under hydrodynamic shear stress involve L-selectin and PSGL-1. A mechanism that amplifies initial leukocyte accumulation of P-selectin in vitro. J. Clin. Invest. 98:1081-1087.
20. Yamamoto, K., Miyagawa, J-I., Hanafusa, T., Itoh, N., Miyazaki, A., Nakawaga, C., Tarui, S., Kono, N., and Matsuzawa, Y. 1992. Endothelial and microvascular abnormalities in the islet of non-obese diabetic mice: transmission and scanning electron microscopic studies. Biomed. Res. 13:259-267.
21. Baron, J. L., Reich, E. P., Visintin, I., and Janeway, C. A. Jr. 1994. The pathogenesis of
   adoptive murine autoimmune diabetes requires an interaction between alpha 4-integrins and vascular cell adhesion molecule-1. J. Clin. Invest. 93:1700-1708.
22. Hanninen, A., Taylor, C., Streeter, P. R., Stark, L. S., Sarte, J. M., Shizuru, J. A., Simell, O., and Michie, S. A. 1993. Vascular addressins are induced on islet vessels during insulitis in nonobese diabetic mice and are involved in lymphoid cell binding to islet endothelium. J. Clin. Invest. 92:2509-2515.
23. Michie, S. A., Sytwu, H. K., McDevitt, J. O., and Yang, X. D. 1998. The roles of alpha 4-integrins in the development of insulin-dependent diabetes mellitus. Curr. Top. Microbiol. Immunol. 231:65-83.
24. Kayo, T., and Koizumi, A. 1998. Mapping of murine diabetogenic gene mody on chromosome 7 at D7Mit258 and its involvement in pancreatic islet and beta cell development during the perinatal period. J. Clin. Invest. 101:2112-2118.
25. Wang, J., Takeuchi, T., Tanaka, S., Kubo, S-K., Kayo, T., Lu, D., Takata, K., Koizumi, A., and Izumi, T. 1999. A mutation in the insulin 2 gene induces diabetes with severe pancreatic beta-cell dysfunction in the Modymouse. J. Clin. Invest. 103:27-37.
26. Dustin, M. L., and Springer, T. A. 1989. T-cell receptor cross-linking transiently stimulates adhesiveness through LFA-1. Nature 341:619-624.
27. Griffiths, E. K., Krawczyk, C., Kong, Y. Y., Raab, M., Hyduk, S. J., Bouchard, D., Chan, V. S., Kozieradzki, I., Oliveira-Dos-Santos, A. J., Wakeham, A., Ohashi, P. S., Cybulsky, M. I., Rudd, C. E., and Penninger, J. M. 2001. Positive regulation of T cell activation and integrin adhesion by the adapter Fyb/Slap. Science 293:2260-2263.
28. Peterson, E. J., Woods, M. L., Dmowski, S. A., Derimanov, G., Jordan, M. S., Wu, J. N., Myung, P. S., Liu, Q. H., Pribila, J. T., Freedman, B. D., Shimizu, Y., and Koretzky, G. A. 2001. Coupling of the TCR to integrin activation by Slap-130/Fyb. Science 293:2263-2265.
29. Campbell, J. J., Hedrick, J., Zlotnik, A., Siani, M. A., Thompson D. A., and Butcher, E. C. 1998. Chemokines and the arrest of lymphocytes rolling under flow conditions. Science 279:381-384.
30. von Andrian, U. H., and Mackay, C. R. 2000. T-cell function and migration. Two sides of the same coin. N. Engl. J. Med. 343:1020-1034.
31. Berlin, C., Bargatze, R. F., Campbell, J. J., von Andrian, U. H., Szabo, M. C., Hasslen, S. R., Nelson, R. D., Berg, E. L., Erlandsen, S. L., and Butcher, E. C. 1995. Alpha 4 integrins mediate lymphocyte attachment and rolling under physiologic flow. Cell 80:413-422.
32. Butcher, E. C., Williams, M., Youngman, K., Rott, L., and Briskin, M. 1999. Lymphocyte trafficking and regional immunity. Adv. Immunol. 72:209-253.
33. Moser, B., and Loetscher, P. 2001. Lymphocyte traffic control by chemokines. Nature Immunol. 2:123-128.
34. Gunn, M. D., Kyuwa, S., Tam, C., Kakiuchi, T., Matsuzawa, A., Williams, L. T., and Nakano, H. 1999. Mice lacking expression of secondary lymphoid organ chemokine have
   defects in lymphocyte homing and dendritic cell localization. J. Exp. Med. 189:451-460.
35. Ngo, V. N., Tang, H. L., and Cyster, J. G. 1998. Epstein-Barr virus-induced molecule 1 ligand chemokine is expressed by dendritic cells in lymphoid tissues and strongly attracts naive T cells and activated B cells. J. Exp. Med. 188:181-191.
36. Constantin, G., Majeed, M., Giagulli, G., Piccio, L., Kim, J. Y., Butcher, E. C., and Laudanna, C. 2000. Chemokines trigger immediate beta 2 integrin affinity and mobility changes: differential regulation and roles in lymphocyte arrest under flow. Immunity 13:759-769.
37. Stein, J. V., Rot, A., Luo, Y., Narasimhaswamy, M., Nakano, H., Gunn, M. D., Matsuzawa, A., Quackenbush, E. J., Dorf, M. E., and von Andrian, U. H. 2000. The CC chemokine thymus-derived chemotactic agent 4 (TCA-4, secondary lymphoid tissue chemokine, 6Ckine, exodus-2) triggers lymphocyte function-associated antigen 1-mediated arrest of rolling T lymphocytes in peripheral lymph node high endothelial venules. J. Exp. Med. 191:61-76.
38. Baekkevold, E. S., Yamanaka, T., Palframan, R. T., Carlsen, H. S., Reinholt, F. P., von Andrian, U. H., Brandtzaeg, P., and Haraldsen, G. 2001. The CCR7 ligand ELC (CCL19) is transcytosed in high endothelial venules and mediates T cell recruitment. J. Exp. Med. 193:1105-1112.
39. Chen, S. C., Vassileva, G., Kinsley, D., Holzmann, S., Manfra, D., Wiekowski, M. T.,
   Romani, N., and Lira, S. A. 2002. Ectopic expression of the murine chemokines CCL21a and CCL21b induces the formation of lymph node-like structures in pancreas, but not skin, of transgenic mice. J. Immunol. 168:1001-1008.
40. Fan, L., Reilly, C. R., Luo, Y., Dorf, M. E., and Lo, D. 2000. Cutting edge: ectopic expression of the chemokine TCA4/SLC is sufficient to trigger lymphoid neogenesis. J. Immunol. 164:3955-3959.
41. Shimizu, Y., Van Seventer, G. A., Horgan, K. J., and Shaw, S. 1990. Regulated expression and binding of three VLA (beta 1) integrin receptors on T cells. Nature 345:250-253.
42. Elices, M. J., Osborn, L., Takada, Y., Crouse, C., Luhowskyj, S,. Hemler, M. E., and Lobb, R. R. 1990. VCAM-1 on activated endothelium interacts with the leukocyte integrin VLA-4 at a site distinct from the VLA-4/fibronectin binding site. Cell 60:577-584.
43. Faull, R. J., Kovach, N. L., Harlan, J. M., and Ginsberg, M. H. 1994 Stimulation of integrin-mediated adhesion of T lymphocytes and monocytes: two mechanisms with divergent biological consequences. J. Exp. Med. 179:1307-1316.
44. Fabien, N., Bergerot, I., Orgiazzi, J., and Thivolet, C. 1996. Lymphocyte function associated antigen-1, integrin alpha 4, and L-selectin mediate T-cell homing to the pancreas in the model of adoptive transfer of diabetes in NOD mice. Diabetes 45:1181-1186.
45. Hanninen, A., Jaakkola, I., and Jalkanen, S. 1998. Mucosal addressin is required for the development of diabetes in nonobese diabetic mice. J. Immunol. 160:6018-6025.
46. Gromme, M., Uytdehaag, F. G., Janssen, H., Calafat, J., van Binnendijk, R. S., Kenter, M. J., Tulp, A., Verwoerd, D., and Neefjes. J. 1999. Recycling MHC class I molecules and endosomal peptide loading. Proc. Natl. Acad. Sci USA. 96:10326-10331.
47. Castellino, F., Boucher, P. E., Eichelberg, K., Mayhew, M., Rothman. J. E., Houghton, A. N., and Germain, R. N. 2000. Receptor-mediated uptake of antigen/heat shock protein
   complexes results in major histocompatibility complex class I antigen presentation via two distinct processing pathways. J. Exp Med. 191:1957-1964.
48. Norbury, C. C., Princiotta, M. F., Bacik, I., Brutkiewicz, R. R., Wood, P., Elliott, T., Bennink, J. R., and Yewdell, J. W. 2001. Multiple antigen-specific processing pathways for activating naive CD8⁺ T cells in vivo. J. Imunol. 166:4355-4362.
49. Kreisel. D., Krupnick, A. S., Gelman, A. E., Engels, F. H., Popma, S. H., Krasinskas, A. M., Balsara, K. R., Szeto, W. Y., Turka, L. A., and Rosengard, B. R. 2002.
   Nonhematopoietic
   allograft cells directly activate CD8⁺ T cells and trigger acute rejection: an alternative mechanism of allorecognition. Nat. Med. 8:233-239.
50. Limmer, A., Ohl, J., Kurts, C., Ljunggren, H. G., Reiss, Y., Groettrup, M., Momburg, F., Arnold, B., and Knolle, P. A. 2000. Efficient presentation of exogenous antigen by liver endothelial cells to CD8⁺ T cells results in antigen-specific T-cell tolerance. Nat. Med. 12:1348-1354.
51. Heath, W. R., and Carbone, F. R. 2001. Cross-presentation, dendritic cells, tolerance and immunity. Annual Rev. Immunol. 19:47-64.
52. Hjelmstrom, P., Fjell, J., Nakagawa, T., Sacca, R., Cuff, C. A., and Ruddle, N. H. 2000. Lymphoid tissue homing chemokines are expressed in chronic inflammation. Am. J. Pathol. 156:1133-8.
53. Luther, S. A., Lopez, T., Bai, W., Hanahan, D., and Cyster, J. G. 2000. BLC expression in pancreatic islets causes B cell recruitment and lymphotoxin-dependent lymphoid neogenesis. Immunity. 12:471-81.
54. Valujskikh, A., Lantz, O., Celli, S., Matzinger, P., and Heeger, P. S. 2002. Crossprimed
   CD8(+) T cells mediate graft rejection via adistinct effector pathway. Nat. Immunol. 3:844-51.

### Incorporation by Reference

All publications and patents mentioned herein are hereby incorporated by reference in their entirety as if each individual publication or patent was specifically and individually indicated to be incorporated by reference.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of modulating homing of T cells to the pancreas comprising contacting the cells with an agonist or an antagonist of the chemokine CCL21, in an amount sufficient to modulate homing of T cells to the pancreas.

2. The method of claim 1, wherein the agonist or antagonist modulates the function of CCL21.

3. The method of claim 2, wherein the agonist or antagonist modulates CCL21 activity.

4. The method of claim 2, wherein the agonist or antagonist modulates CCL21 expression.

5. The method of claim 3, wherein the agonist or antagonist modulates the interaction between CCL21 and a chemokine receptor of the T cells.

6. The method of claim 5, wherein the chemokine receptor is CCR7.

7. The method of claim 5, wherein the chemokine receptor is CXCR3.

8. The method of claim 3, wherein the agonist or antagonist is an antibody against CCL21.

9. The method of claim 3, wherein the agonist or antagonist is a mutated form or a mimic of CCL21.

10. The method of claim 3, wherein the agonist or antagonist is a peptidomimetic.

11. A method of modulating homing of T cells to the pancreas comprising contacting the cells with an agonist or an antagonist of a chemokine receptor of the T cells, in an amount sufficient to modulate homing of T cells to the pancreas.

12. The method of claim 11, wherein the agonist or antagonist modulates the function of the chemokine receptor.

13. The method of claim 12, wherein the agonist or antagonist modulates the chemokine receptor activity.

14. The method of claim 12, wherein the agonist or antagonist modulates the chemokine receptor expression.

15. The method of claim 13, wherein the agonist or antagonist modulates the interaction between the chemokine receptor and CCL21.

16. The method of claim 11, wherein the chemokine receptor is CCR7.

17. The method of claim 11, wherein the chemokine receptor is CXCR3.

18. The method of claim 13, wherein the agonist or antagonist is an antibody against the chemokine receptor.

19. The method of claim 13, wherein the agonist or antagonist is a mutated form or a mimic of the chemokine receptor.

20. The method of claim 13, wherein the agonist or antagonist is a peptidomimetic.

21. A method of treating an individual suffering from insulin-dependent diabetes, comprising administering to the individual a therapeutically effective amount of an antagonist of the chemokine CCL21, wherein the antagonist blocks homing of T cells to the pancreas and thereby prevents damage to the insulin-producing β cells.

22. The method of claim 21, wherein the agonist or antagonist modulates the function of CCL21.

23. The method of claim 22, wherein the antagonist inhibits CCL21 activity.

24. The method of claim 22, wherein the antagonist inhibits CCL21 expression.

25. The method of claim 23, wherein the antagonist inhibits the interaction between CCL21 and a chemokine receptor of the T cells.

26. The method of claim 25, wherein the chemokine receptor is CCR7.

27. The method of claim 25, wherein the chemokine receptor is CXCR3.

28. The method of claim 23, wherein the antagonist is an antibody against CCL21.

29. The method of claim 23, wherein the antagonist is a mutated form of CCL21 or a CCL21 mimic.

30. The method of claim 23, wherein the antagonist is a peptidomimetic.

31. The method of claim 21, wherein the antagonist of the chemokine CCL21 is administered with another compound for treating insulin-dependent diabetes.

32. The method of claim 31, wherein the compound is insulin.

33. A method of treating an individual suffering from insulin-dependent diabetes, comprising administering to the individual a therapeutically effective amount of an antagonist of a chemokine receptor of the T cells, wherein the antagonist blocks homing of T cells to the pancreas and thereby prevents damage to the insulin-producing β cells.

34. The method of claim 33, wherein the agonist or antagonist modulates the function of the chemokine receptor.

35. The method of claim 34, wherein the antagonist inhibits the chemokine receptor activity.

36. The method of claim 34, wherein the antagonist inhibits the chemokine receptor expression.

37. The method of claim 35, wherein the antagonist inhibits the interaction between the chemokine receptor and CCL21.

38. The method of claim 33, wherein the chemokine receptor is CCR7.

39. The method of claim 33, wherein the chemokine receptor is CXCR3.

40. The method of claim 35, wherein the antagonist is an antibody against the chemokine receptor.

41. The method of claim 35, wherein the antagonist is a mutated form or a mimic of the chemokine receptor.

42. The method of claim 35, wherein the antagonist is a peptidomimetic.

43. The method of claim 33, wherein the antagonist of the chemokine receptor is administered with another compound for treating insulin-dependent diabetes.

44. The method of claim 43, wherein the compound is insulin.

45. A method of modulating homing of T cells to the pancreas in an individual, comprising administering to the individual an agonist or an antagonist of the chemokine CCL21 in an amount sufficient to modulate homing of T cells to the pancreas.

46. A method of modulating homing of T cells to the pancreas in an individual, comprising administering to the individual an agonist or an antagonist of a chemokine receptor in an amount sufficient to modulate homing of T cells to the pancreas.

47. A method of preventing or reducing the onset of insulin-dependent diabetes in an individual, comprising administering to the individual an effective amount of an antagonist of CCL21, wherein the antagonist is effective to prevent or reduce the onset of insulin-dependent diabetes.

48. A method of preventing or reducing the onset of insulin-dependent diabetes in an individual, comprising administering to the individual an effective amount of an antagonist of a chemokine receptor of the T cells, wherein the antagonist is effective to prevent or reduce the onset of insulin-dependent diabetes.
